(19) United States
(12) Patent Application Publication
(10) Pub. No.: US 2013/0209386 A1
(43) Pub. Date: Aug. 15, 2013

(54) NEW USES

(71) Applicants: Jonathan Cove, Wetherby (GB); Daniel Fitzgerald, Wetherby (GB); Nicholas Read, Wetherby (GB)

(72) Inventors: Jonathan Cove, Wetherby (GB); Daniel Fitzgerald, Wetherby (GB); Nicholas Read, Wetherby (GB)

(73) Assignee: EVOCUTIS PLC, Wetherby (GB)

(21) Appl. No.: 13/812,232

(22) PCT Filed: Jul. 25, 2011
(86) PCT No.: PCT/GB2011/051414

§ 371 Date: Mar. 19, 2013

(30) Foreign Application Priority Data
Jul. 28, 2010 (GB) 1012589.6
Jun. 2, 2011 (GB) 1109229.3

(51) Int. Cl. A61K 33/34; A61K 31/30; A61K 33/24; A61Q 15/00; A61K 8/58; A61K 8/23; A61K 8/20; A61K 31/122; A61K 31/28

(52) U.S. Cl. CPC A61K 33/34; A61K 31/122; A61K 31/30; A61K 33/245; A61K 31/28; A61K 8/58; A61K 8/23; A61K 8/20; A61Q 15/00
USPC 424/65; 424/637; 514/500; 424/634; 424/632; 424/653; 514/492; 514/495

(57) ABSTRACT

An antibacterial or anti-acne formulation containing (a) tropone or a substituted tropone and (b) a metal or metal salt selected from copper, copper salts, bismuth, bismuth salts, silver, silver salts, and mixtures thereof, for use in the treatment of either acne or body odour. The substituted tropone may be for example tropolone or hinokitiol.

### FIELD OF THE INVENTION

This invention relates to new uses for certain combinations of compounds, as antibacterial and anti-acne agents.

### BACKGROUND TO THE INVENTION

Tropolone, also known as 2-hydroxy-2,4,6-cycloheptatrien-1-one, 2-hydroxytropone and purpurocatechol, is a derivative of tropone (cyclohepta-2,4,6-trien-1-one). Tropone, tropolone and related compounds are known to exert a variety of biological effects including anti-tumour activity, phytogrowth-inhibitory activity, antimicrobial activity against bacteria and fungi, insecticidal activity, and inhibition of metalloproteinases (see for example Morita Y et al, 2003 Biol Pharm Bull 26(10): 1487-1490, and Trust T J, 1975, Antimicrobial Agents and Chemotherapy 7(5): 500-506). Further properties of the tropolonoids, including their antibacterial activity, are described in “A fresh look at natural tropolonoids” by Bentley R, 2008 Natural Product Reports 25: 118-138.

Tropolone has also been used in personal care products as a skin conditioning agent, and has been incorporated into products such as moisturisers, sunscreens, exfoliants, anti-wrinkle formulations and shampoos. Tropolonoids such as tropolone and hinokitiol (4-isopropyltropolone) have been used as antidandruff agents, including in combination with metal salts, whilst hinokitiol has been reported to have use in promoting hair growth (Hwang & Kim, Journal of Microencapsulation, August 2008, 25(5): 351-356). Nomiya et al, in Journal of Inorganic Biochemistry, 98 (2004): 46-60, have described complexes of hinokitiol with silver (I), aluminium (III) and cobalt (II), and their antimicrobial activities. WO-2010/055850 discloses an antiviral foam hand wash containing a polyalkylene glycol ether and hinokitiol or a metal complex thereof JP-2009-84265 describes the use of metal-tropolone complexes in anti-Legionella products. JP-2001-40222 describes the incorporation of antimicrobial metal components and tropolone-based compounds into antimicrobial polymers.

Certain metal salts have also been used as antimicrobial and anti-acne agents. Copper and bismuth salts, for example, have been utilised as antimicrobial agents, including in anti-acne formulations—see U.S.-2005/0123620 and U.S.-2008/0020059 which refer to the use of various polyvalent metal compounds, including copper and bismuth salts, in the topical treatment of acne and warts; U.S. Pat. No. 6,294,186 which describes a topical antimicrobial composition for the treatment of acne containing a benzoic acid analogue and a metal salt which can be a copper salt such as a halide, sulphate or salicylate; and EP-1 437 124 which describes a topical anti-acne formulation containing a hydroxyacid, a copper salt such as a sulphate or pidolate, a zinc salt, an algae extract and a haloalkynyl carbamate.

In WO-2007/096601, copper salts are used in combination with quinones, in particular benzo- and hydroquinones, as antimicrobial and anti-acne agents. This document demonstrates the activity of the combinations against propionibacteria such as P. acnes (the bacteria involved in inflammatory acne) and staphylococci.

WO-96/37228 describes the use of bismuth salts as antibacterial agents in wound treatment compositions, together with wound healing agents. EP-1 702 621 discloses antimicrobial combinations of thiol-containing complexing agents and bismuth salts: these compositions can be used, for example, as surface disinfectants, topical pharmaceuticals and antimicrobial soaps, such as to treat microbial skin conditions. WO-01/00151 also describes combining a pyrithione with a metal ion source, which can be a bismuth salt, in a topical antimicrobial formulation in particular for antifungal and antidandruff use. In WO-2008/035085, bismuth salts are shown to be active against propionibacteria, and thus to be of use in the treatment of acne.

Certain silver salts have also been recognised as antimicrobial agents, and have been used in wound care formulations.

It has now surprisingly been found that when a tropolonoid such as tropolone is combined with certain types of metal or metal salt, a synergistic effect can be observed on their combined level of antibacterial activity, including against P. acnes and against the coryneform bacteria associated with body odour. As a result, novel antibacterial and anti-acne formulations can be prepared, in particular for topical application, either with improved efficacy and/or containing lower levels of at least one of the active ingredients than would previously have been thought necessary.

### STATEMENTS OF THE INVENTION

According to a first aspect of the present invention there is provided an antibacterial or anti-acne formulation containing (a) tropone or a substituted tropone and (b) a metal or metal salt selected from copper, copper salts, bismuth, bismuth salts, silver, silver salts, and mixtures thereof, for use in the treatment of either acne or body odour.

A substituted tropone may carry one or more substituents, which may be attached to the oxygen atom of the C═O group or more typically to carbon atoms in the cycloheptene ring. Such substituents may for example be selected from hydroxyl; alkyl and alkenyl groups; the carboxylic acid group —CO₂H or an optionally cyclic acid anydride; alkoxyl groups (in particular C₁ to C₆ or C₁ to C₄ alkoxyl groups such as methoxyl or ethoxyl, more particularly methoxyl); halogens such as fluoro, chloro or bromo, in particular chloro; nitro groups —NO₂; amino groups —NR₂ (where each R is independently either hydrogen or hydrocarbyl, suitably either hydrogen or C₁ to C₆ alkyl, more suitably either hydrogen or C₁ to C₄ alkyl, for example either hydrogen, methyl or ethyl), in particular NH₂; and in cases amide groups such as —NR—COR or —NH—COR where R is as defined above.

An alkyl or alkenyl substituent may in particular be an alkyl substituent. It may be selected from C₁ to C₆ or C₁ to C₄ alkyl or alkenyl groups, for instance methyl, ethyl, n-propyl, isopropyl, n-butyl, isobutyl or t-butyl groups, more particularly methyl, ethyl or isopropyl groups. It may itself carry one or more substituents such as those listed above; however, in an embodiment it is unsubstituted.

The tropone may in particular be substituted with one or more groups selected from hydroxyl, alkoxyl and alkyl groups. It may be substituted with one or more groups selected from hydroxyl and alkyl groups. In an embodiment, it is selected from tropolone and substituted tropolones, wherein a substituted tropolone carries one or more substituents selected from those listed above, in particular alkyl groups.

A substituted tropone may be a bicyclic tropone or tropolone, which is fused to another typically 5- or 6- or 7-membered ring. The additional ring may itself carry one or more substituents, for example of the type listed above; it may contain one or more heteroatoms such as sulphur, nitrogen and/or oxygen. The additional ring may itself be fused to another ring, so that the substituted tropone is a tricyclic molecule, or in cases a tetra-, penta- or hexacyclic moiety. In cases however it may be preferred for the substituted tropone not to be multicyclic, or for the substituted tropone to be either mono- or bicyclic.

In an embodiment of the invention, the tropone is mono-substituted. In an embodiment, it is substituted at the 2-position. In an embodiment, it is substituted with one hydroxyl group. Thus, the substituted tropone (a) may be tropolone.

In an embodiment, the tropone carries more than one substituent (for example it may be di- or tri-substituted). In such a case, one of the substituents may be a hydroxyl group, which may occupy the 2-position; the tropone may therefore be a substituted tropolone such as a hydroxytropolone (for example hydroxytropolone itself, 7-hydroxy-3-isopropyltropolone, 7-hydroxy-4-isopropyltropolone, 2-hydroxy-4-isopropyltropolone or 7-hydroxy-3-isopropenyltropolone) or dihydroxytropolone.

Where the tropone carries more than one substituent, one of the substituents may be an alkoxyl group, in particular a methoxyl group, as in for example pygmaein and isopygmaein.

Where the tropone carries more than one substituent, one of the substituents may be an alkyl group. An alkyl group may be a C₁ to C₄ alkyl group such as isopropyl, which may occupy the 4-position. Thus, the substituted tropone may be hinokitiol (also known as β-thujaplicin, 2-hydroxy-4-isopropyl-2,4,6-cycloheptatrien-1-one or 4-isopropyltropolone) or another thujaplicin.

The substituted tropone may be a hydroxytropolone acid or derivative thereof, such as stipitatic acid, stipitatonic acid, stipitalide, stipitaldehydic acid, puberulonic acid or puberulic acid.

In an embodiment, the substituted tropone is selected from tropolone, hinokitiol and mixtures thereof. In an embodiment, it is tropolone. In an embodiment, it is hinokitiol. In certain cases, however, it may be preferred for the substituted tropone not to be hinokitiol.

According to the invention, the tropone or substituted tropone is combined with a metal or metal salt selected from copper, copper salts, bismuth, bismuth salts, silver, silver salts, and mixtures thereof. In an embodiment, it is combined with a metal or metal salt selected from copper, copper salts, bismuth, bismuth salts, and mixtures thereof. In an embodiment, it is combined with a metal or metal salt selected from copper, copper salts, and mixtures thereof. In another embodiment, it is combined with a metal or metal salt selected from bismuth, bismuth salts, and mixtures thereof. In yet another embodiment it is combined with a metal or metal salt selected from silver, silver salts, and mixtures thereof. In certain embodiments, in particular when the optionally substituted tropone (a) is hinokitiol, it may be combined with a metal or metal salt selected from bismuth, bismuth salts, silver, silver salts, and mixtures thereof.

In an embodiment, the optionally substituted tropone is combined with a metal salt selected from copper salts, bismuth salts, silver salts, and mixtures thereof. In an embodiment, it is combined with a metal salt selected from copper salts, bismuth salts, and mixtures thereof. In an embodiment, it is combined with a copper salt or mixture of copper salts.

In an embodiment, the metal or metal salt is suitable for cosmetic use. In an embodiment, it is suitable for pharmaceutical (which includes veterinary) use.

In the context of the present invention, the term “copper salt” includes copper (I), (II) and (III) salts, as well as copper (I), (II) or (III) complexes. In an embodiment a copper salt in a formulation for use according to the invention is a copper (I) (cuprous) or copper (II) (cupric) salt, in particular a copper (II) salt.

In a formulation for use according to the invention, a copper salt may for instance be selected from copper carboxylates; copper halides; copper sulphadiazine; copper sulphate (in particular the pentahydrate); copper nitrate; copper carbonate; copper carbonate hydroxide; copper oxide; copper oxychloride; copper hydroxide; copper acetylacetonate; copper PCA (pyrrolidone carboxylic acid); copper PCA methylsilanol; copper acetyl tyrosinate methylsilanol; copper acetylmethionate; copper aminoacetylamidoimidazolyl propionate; copper picolinate; copper peptides such as copper tripeptide-1, bis(tripeptide-1) copper acetate, copper ascorbyl phosphate succinoyl tripeptide-34 and alanine/histidine/lysine copper polypeptide HCl; copper ATP; copper DNA; copper amino acid salts (eg copper glutamate, copper aspartate and copper glycinate); copper silicates; copper salts of quinolines—especially hydroxyquinolines—and their derivatives (eg the copper salt of 8-hydroxyquinoline); copper pyrithione and other copper salts of pyridine thiols; copper phosphates, including for example sodium calcium copper phosphate and copper pyridoxal-5-phosphate; chlorophyllin copper complexes such as sodium copper chlorophyllin; copper chlorophyll; disodium EDTA copper; and mixtures thereof.

Suitable copper carboxylates include lactate, citrate (including for example disodium cupric citrate, and copper (II) citrate dihydrate), ascorbate, acetate (either copper (I) acetate or copper (II) acetate), gluconate, laurate, myristate, palmitate, salicylate, aspirinate, stearate, succinate, tartrate, undecylenate, usnate (in particular copper (II) di-usnate), neodecanoate and ricinoleate. A carboxylate may be for example a C2 to C20, a C2 to C18, a C2 to C16, a C2 to C14, a C2 to C12, a C2 to C10, a C2 to C8 or a C2 to C6 carboxylate.

Suitable halides include copper chloride, copper bromide and copper iodide, for example copper (II) chloride or copper (I) iodide.

In an embodiment of the invention, the copper salt may be selected from copper sulphate, copper acetate, copper glycinate, copper gluconate, copper acetylmethionate, copper picolinate, copper citrate, copper PCA methylsilanol, copper acetyl tyrosinate methylsilanol, copper tripeptide-1, copper ATP, copper aspartate, copper DNA, copper carbonate hydroxide, copper pyridoxal-5-phosphate, cupric chloride, disodium cupric citrate, disodium EDTA-copper, bis(tripeptide-1) copper acetate, copper ascorbyl phosphate succinoyl tripeptide-34, chlorophyllin copper complexes, copper aminoacetylamidoimidazolyl propionate, copper chlorophyll; sodium calcium copper phosphate, alanine/histidine/lysine copper polypeptide HCl, and mixtures thereof. In each case the salt is preferably the copper (II) salt.

In another embodiment, the copper salt may be selected from copper sulphate, copper acetate, copper acetylmethionate, copper PCA, disodium EDTA-copper, chlorophyllin copper complexes (for example sodium copper chlorophyllin), copper chlorophyll, and mixtures thereof.

In a further embodiment, the copper salt may be selected from copper sulphate, copper acetate, copper PCA and mixtures thereof.

In particular where the formulation is for use in the treatment of acne, the copper salt may be selected from:
(a) copper (II) sulphate (in particular the pentahydrate), copper (I) acetate, copper (I) iodide, copper (II) acetate, copper (II) acetylmethionate, copper (II) carbonate basic, copper (II) chloride, copper (II) citrate dihydrate, copper (II) gluconate, copper (II) PCA, copper (II) picolinate, copper (II) salicylate, copper (II) silicate, copper (II) usnate, EDTA copper (II) disodium salt, sodium copper (II) chlorophyllin, and mixtures thereof; or
(b) copper (II) sulphate (in particular the pentahydrate), copper (I) acetate, copper (I) iodide, copper (II) acetate, copper (II) acetylmethionate, copper (II) carbonate basic, copper (II) chloride, copper (II) citrate dihydrate, copper (II) gluconate, copper (II) PCA, copper (II) salicylate, copper (II) silicate, copper (II) usnate, EDTA copper (II) disodium salt, sodium copper (II) chlorophyllin, and mixtures thereof; or
(c) copper (II) sulphate (in particular the pentahydrate), copper (II) acetate, copper (II) acetylmethionate, copper (II) carbonate basic, copper (II) gluconate, copper (II) PCA, copper (II) salicylate, copper (II) usnate, EDTA copper (II) disodium salt, sodium copper (II) chlorophyllin, and mixtures thereof; or
(d) copper (II) sulphate (in particular the pentahydrate), copper (II) acetate, copper (II) acetylmethionate, copper (II) carbonate basic, copper (II) PCA, EDTA copper (II) disodium salt, sodium copper (II) chlorophyllin, and mixtures thereof; or
(e) copper (II) sulphate (in particular the pentahydrate), copper (II) acetylmethionate, EDTA copper (II) disodium salt, sodium copper (II) chlorophyllin, and mixtures thereof.

In another embodiment, the copper salt is selected from copper (II) sulphate (in particular the pentahydrate), copper (I) acetate, copper (I) iodide, copper (II) acetate, copper (II) acetylmethionate, copper (II) carbonate basic, copper (II) chloride, copper (II) citrate dihydrate, copper (II) PCA, copper (II) salicylate, copper (II) silicate, copper (II) usnate, EDTA copper (II) disodium salt, sodium copper (II) chlorophyllin, and mixtures thereof.

In an embodiment, the copper salt is selected from copper (II) sulphate (in particular the pentahydrate), copper (I) acetate, copper (I) iodide, copper (II) acetate, copper (II) acetylmethionate, copper (II) carbonate basic, copper (II) chloride, copper (II) citrate dihydrate, copper (II) salicylate, copper (II) silicate, copper (II) usnate, EDTA copper (II) disodium salt, and mixtures thereof.

In an embodiment, the copper salt is selected from copper (II) sulphate (in particular the pentahydrate), copper (II) acetate, copper (II) chloride, copper (II) citrate dihydrate, copper (II) silicate, copper (II) usnate, EDTA copper (II) disodium salt, and mixtures thereof.

In an embodiment, the copper salt is selected from copper (II) sulphate (in particular the pentahydrate), copper (II) acetate, copper (II) acetylmethionate, copper (II) carbonate basic, copper (II) salicylate, copper (II) usnate, EDTA copper (II) disodium salt, and mixtures thereof.

In an embodiment, the copper salt is selected from copper (II) sulphate (in particular the pentahydrate), copper (II) acetate, copper (II) acetylmethionate, copper (II) carbonate basic, EDTA copper (II) disodium salt, and mixtures thereof.

In yet another embodiment, the copper salt is selected from copper sulphate (in particular the pentahydrate), copper PCA and mixtures thereof. In an embodiment, it is copper sulphate. In an embodiment, it may be preferred for the copper salt not to be copper (II) picolinate. In an embodiment, in particular when the formulation is for use in the treatment of body odour, it may be preferred for the copper salt not to be copper (II) usnate or sodium copper (II) chlorophyllin.

In a further embodiment, the copper salt is selected from copper sulphate (in particular the pentahydrate); copper halides (for example copper iodide or copper chloride); copper carbonate; copper carboxylates (for example copper acetate, copper citrate dihydrate, copper (II) D-gluconate, copper (II) salicylate or copper usnate); copper (II) glycinate; copper (II) silicate; copper (II) PCA; sodium copper (II) chlorophyllin; copper (II) picolinate; and mixtures thereof.

In a yet further embodiment, the copper salt is selected from copper sulphate (in particular the pentahydrate); copper halides (for example copper iodide or copper chloride); copper carbonate; copper carboxylates (for example copper acetate or copper citrate dihydrate); copper (II) D-gluconate; copper (II) salicylate; copper (II) silicate; copper (II) PCA; copper (II) picolinate; and mixtures thereof.

In particular where the formulation is for use in the treatment of body odour, the copper salt may be selected from:
(a) copper (II) sulphate, copper (II) iodide, copper (II) acetate, copper (I) acetate, copper (II) D-gluconate, copper (II) salicylate, copper (II) silicate, copper (II) PCA, copper (II) chloride, copper (II) citrate dihydrate, copper (II) glycinate, copper (II) carbonate, copper (II) picolinate, and mixtures thereof; or
(b) copper (II) sulphate, copper (II) iodide, copper (II) acetate, copper (I) acetate, copper (II) D-gluconate, copper (II) salicylate, copper (II) silicate, copper (II) PCA, copper (II) chloride, copper (II) citrate dihydrate, copper (II) carbonate, and mixtures thereof; or
(c) copper (II) sulphate, copper (I) acetate, copper (II) salicylate, and mixtures thereof.

In an embodiment, it may be preferred for the copper salt not to be a complex formed between a copper ion and a complexing agent, in particular a bi- or multi-dentate ligand.

In the context of the present invention, the term “bismuth salt” includes bismuth (III) and (V) salts, as well as potentially bismuth (III) and (V) complexes. In an embodiment, the bismuth salt is a bismuth (III) salt.

A bismuth salt may for instance be selected from bismuth carboxylates, bismuth halides, bismuth sulphadiazine, bismuth sulphate, bismuth nitrate, bismuth subnitrate, bismuth carbonate, bismuth subcarbonate, bismuth oxide, bismuth oxychloride, bismuth hydroxide, bismuth phosphate, bismuth aluminate, bismuth tribromophenate, bismuth thiol, bismuth peptides, bismuth salts of quinolines and their derivatives (eg bismuth hydroxyquinolines), bismuth pyrithione and other bismuth salts of pyridine thiols, bismuth amino acid salts such as the glycinate, tripotassium dicitrato bismuthate, and mixtures thereof. It may be selected from bismuth citrate, bismuth subgallate, bismuth chloride oxide, bismuth nitrate, bismuth subnitrate and mixtures thereof.

A bismuth salt may be a basic bismuth salt (bismuth subsalt) such as the subsalts referred to above.

Suitable bismuth carboxylates include the salicylate, subsalicylate, lactate, citrate, subcitrate, ascorbate, acetate, dipropylacetate, tartrate, sodium tartrate, gluconate, subgallate, benzoate, laurate, myristate, palmitate, propionate, stearate, undecylenate, aspirinate, neodecanoate and ricinoleate. Of these, basic bismuth salicylate (bismuth subsalicylate) and bismuth citrate may be preferred. A carboxylate may be for example a C2 to C20, a C2 to C18, a C2 to C16, a C2 to C14, a C2 to C12, a C2 to C10, a C2 to C8 or a C2 to C6 carboxylate.

Suitable halides include bismuth chloride, bismuth bromide and bismuth iodide, in particular bismuth chloride.

In an embodiment, the bismuth salt may be selected from bismuth halides (in particular bismuth chloride), bismuth nitrates, bismuth carboxylates, and mixtures thereof. In another embodiment it may be selected from bismuth subsalicylate, bismuth salicylate, bismuth subgallate, bismuth subcitrate, bismuth citrate, bismuth acetate, bismuth nitrate, bismuth subnitrate and mixtures thereof. In yet another embodiment it may be selected from bismuth subsalicylate, bismuth citrate, bismuth subnitrate and mixtures thereof; or from bismuth subsalicylate, bismuth subnitrate and mixtures thereof; or from bismuth subsalicylate, bismuth citrate and mixtures thereof.

In an embodiment, the bismuth salt may be selected from bismuth carboxylates (in particular bismuth acetates, bismuth citrates and bismuth gallates), bismuth nitrates, bismuth halides (in particular bismuth chloride), and mixtures thereof. In an embodiment, it is selected from bismuth acetate, bismuth citrate, bismuth subgallate, bismuth subnitrate, bismuth chloride and mixtures thereof. In an embodiment, it is selected from bismuth acetate, bismuth subgallate, bismuth chloride and mixtures thereof.

In the context of the present invention, the term “silver salt” includes silver complexes. It may also include silver-containing multiple salts, for example ammonium silver salts, sodium calcium silver salts, sodium magnesium silver salts, sodium silver aluminium salts, ammonium silver zinc aluminium salts, silver copper salts and silver magnesium aluminium salts.

A silver salt may for instance be selected from carboxylates, silicates, borosilicates, acetylmethionates, halides (for example chlorides), phosphates, nitrates, oxides, sulphates, and mixtures thereof. In an embodiment it may be a zeolite salt such as ammonium silver zeolite or silver copper zeolite.

Suitable silver carboxylates include the citrate, lactate and acetate. A carboxylate may be for example a C2 to C20, a C2 to C18, a C2 to C16, a C2 to C14, a C2 to C12, a C2 to C10, a C2 to C8 or a C2 to C6 carboxylate

In an embodiment, the silver salt is selected from ammonium silver zeolite, ammonium silver zinc aluminium silicate, silver acetylmethionate, silver bicarbonate, silver borosilicate, silver chloride, silver acetate, silver citrate (in particular the hydrate), silver copper zeolite, silver lactate, silver magnesium aluminium phosphate, silver nitrate, silver oxide, silver sulphate, sodium calcium silver phosphate, sodium magnesium silver phosphate, sodium silver aluminium silicate, and mixtures thereof. In an embodiment, in particular where the formulation is for use in the treatment of body odour, it may be selected from ammonium silver zeolite, ammonium silver zinc aluminium silicate, silver copper zeolite, silver lactate and mixtures thereof.

In an embodiment, the silver salt may be selected from ammonium silver zeolite, ammonium silver zinc aluminium silicate, silver acetylmethionate, silver bicarbonate, silver borosilicate, silver chloride, silver citrate, silver copper zeolite, silver lactate, silver magnesium aluminium phosphate, silver nitrate, silver oxide, silver sulphate, sodium calcium silver phosphate, sodium magnesium silver phosphate, sodium silver aluminium silicate, and mixtures thereof.

In an embodiment, the silver salt is selected from silver carboxylates and mixtures thereof. In an embodiment it is a silver citrate, in particular silver citrate hydrate.

Generally speaking a metal salt for use in the formulation of the invention may be either organic or inorganic. It may be present in an at least partially hydrated form, and may thus be formulated in the presence of an aqueous solvent. Alternatively it may be used in the form of a lipid-soluble salt, suitably in the presence of an organic solvent.

In an embodiment, it may be preferred for the metal salt not to be a complex formed between a metal ion and either tropone or a substituted tropone such as tropolone or hinokitiol. In other words, it may be preferred for the formulation to be prepared using, in addition to the tropone (a), a first metal salt other than a metal-tropolonoid complex, even if a metal-tropolonoid complex is formed in the formulation prior to, or at the point of, use. The formulation will then contain, in addition to the metal-tropolonoid complex formed prior to or at the point of use, an additional anion or complexing agent derived from the first metal salt.

In an embodiment of the invention, the component (b) of the formulation comprises an atomic metal selected from copper, bismuth, silver and mixtures thereof. This may be in a particulate, for example nanoparticulate, form. It may be in the form of a colloidal suspension. It may for example be nanoparticulate copper or colloidal silver. The formulation may be such that the metal forms a metal salt in situ before or on application to a surface such as the skin.

In a formulation for use according to the invention, either or both of the tropone (a) and the metal or metal salt (b) may be present in the form of a suitable derivative. In general a derivative may be a pharmaceutically acceptable (which term includes acceptable for veterinary use) derivative. It may be for example a salt, complex or solvate, or a so-called “prodrug” form or protected form which reverts to an active form of the relevant compound at an appropriate time on or after administration. In an embodiment of the invention, however, the optionally substituted tropone (a) is present in the form of a single, underivatised molecule of the relevant tropone. In an embodiment, the metal or metal salt (b) is present in the form of the underivatised element, or of an underivatised ion, molecule or complex of the relevant species.

The concentration of the optionally substituted tropone in the formulation might suitably be 0.1% w/v or greater, or 0.25 or 0.5 or 0.75 or in cases 1% w/v or greater. Its concentration might be up to 10% w/v, or up to 7.5 or 5 or 3 or 2.5% w/v. Its concentration might for example be from 0.5 to 5% w/v, or from 0.5 to 3% w/v, or from 1 to 3% w/v, such as about 1% w/v or about 2% w/v.

The concentration of the metal or metal salt in the formulation might suitably be 0.01% w/v or greater, or 0.1% w/v or greater. Its concentration might be up to 10% w/v, or up to 2% w/v, such as from 0.1 to 1% w/v.

The weight ratio of the tropone (a) to the metal or metal salt (b) in the formulation may be up to 100:1, for example up to 50:1 or 25:1 or 10:1, or up to 8:1 or 5:1 or 4:1 or 2:1. This ratio may be 1:100 or greater, for example 1:50 or greater or 1:25 or greater or 1:10 or greater, or 1:5 or 1:2 or 1:1 or greater. In cases it may be 2:1 or 5:1 or 10:1 or greater. It may for example be from about 8:1 to 1:1, or from about 4:1 to 1:1, or from about 2:1 to 1:1.

In an embodiment, a formulation for use according to the invention is suitable for topical or local application to the skin, in particular human skin. A formulation which is “suitable for” topical or local application may also be adapted for topical or local application.

A formulation for use according to the invention may be in the form of a fluid, for example a lotion, cream, ointment, varnish, foam, paste, gel or other viscous or semi-viscous fluid, or a less viscous fluid such as might be used in sprays or aerosols. It may take the form of a solution, suspension or emulsion. It may take the form of a powder or of granules, which may be designed to be added to liquid (eg water) prior to use.

In an embodiment the formulation is, or may be, applied to a carrier such as a sponge, swab, brush, pad, tissue, cloth, wipe, skin patch or dressing (which includes a bandage, plaster, skin adhesive or other material designed for application to a tissue surface), to facilitate its administration.

For use in the treatment of acne, the formulation may for example take the form of a lotion, cream, ointment, varnish, foam, paste or gel; or it may be, or be capable of being, applied to a carrier of the type described above.

For use in the treatment of body odour, the formulation may for example take the form of an aerosol, or of a roll-on or “stick” anti-perspirant or deodorant formulation, or of a dusting powder such as a talcum powder, or of a gel or cream or ointment. It may be coated on or incorporated into a sock or shoe, or a shoe insert such as an insole.

A formulation for use according to the invention may be intended for pharmaceutical (which includes veterinary but is preferably human) use, and/or for cosmetic or other non-medical care purposes (for example, to cleanse the skin, or to improve the appearance, feel or smell of the skin).

A formulation for use according to the invention may contain excipients and other additives known for use in pharmaceutical or veterinary formulations. Suitable excipients for use in formulations designed for topical or local application will be well known to those skilled in the art. Those included will depend on the intended mode and site of application for the formulation. In the context of formulations for topical application to the skin, examples may for instance be found in Williams' Transdermal and Topical Drug Delivery (Pharmaceutical Press, 2003) and other similar reference books. See also Date, A A et al, Skin Pharmacol Physiol, 2006, 19(1): 2-16 for a review of topical drug delivery strategies, and also Skin Delivery Systems, 2006, John J Wille, Ed, Blackwell Publishing; Textbook of Cosmetic Dermatology, 2004, 3^{th} edition, Robert Baran, Howard I Maibach, Taylor & Francis; and Skin Care Beyond the Basics, 2001, Mark Lees, Milady.

The optionally substituted tropone and the metal or metal salt may each independently be present in the form of a solution or suspension, the term “suspension” including emulsions, micellar systems and other multi-phase dispersions.

The excipient(s) used may be suitable for targeting or controlling release of the formulation, or of a component of the formulation, at the intended site of administration, for instance to target a desired site and/or time of delivery. Such excipients may for instance target the formulation to a region of the skin, for example the stratum corneum or the pilosebaceous follicles, or to hair follicles. They may delay or otherwise control release of the formulation over a particular time period. Either or both of the tropone (a) and the metal or metal salt (b) may be microencapsulated: suitable encapsulating entities include liposomes, niosomes, aspasomes, cubosomes, microsponges, microemulsions, hydrogels and solid lipid nanoparticles. Particularly suitable liposomes, for topical application to the skin, are those made from stratum corneum lipids, eg ceramides, fatty acids or cholesterol.

Where the formulation is intended for topical application to the skin, examples of suitable additives include emollients, moisturisers, perfumes, antioxidants, preservatives, stabilisers, gelling agents and surfactants; others may be found in Williams' Transdermal and Topical Drug Delivery (see above). For the treatment of acne, however, it may be preferred for the formulation not to contain an emollient.

Such a formulation may further contain additional active agents such as antimicrobial (in particular antibacterial) agents. For example, it may contain one or more agents selected from anti-acne agents, keratolytics, comedolytics, agents capable of normalising keratinocyte and/or sebocyte function, anti-inflammatories, anti-proliferatives, antibiotics, anti-androgens, sebostatic/sebosuppressive agents, anti-pruritics, immunomodulators, agents which promote wound healing, additional antimicrobial agents, sunscreens, skin lightening agents, anti-ageing substances, and mixtures thereof.

In this context an additional antimicrobial agent may be selected from biocides, disinfectants, antiseptics, antibiotics, bacteriophages, enzymes, anti-adhesins, immunoglobulins, antimicrobially active antioxidants, and mixtures thereof; it may be active as a bactericide, in particular against propionibacteria and/or corynebacteria and/or staphylococci. It may however be preferred for the tropone (a) and the metal or metal salt (b) to be the only active agents in the formulation, or at least to be the only antimicrobially or antibacterially active agents and/or the only anti-acne active agents.

In particular where the formulation is for use in the treatment of body odour, the formulation may contain one or more additional agents selected from additional antimicrobial agents, deodorants, anti-perspirants, perfumes, and mixtures thereof. In this context an additional antimicrobial agent may be active as a bactericide, in particular against coryneform bacteria.

In certain cases it may be preferred for the formulation not to contain a polymer, in particular a solid polymer such as a resin.

A formulation for use according to the invention may be incorporated into, and hence applied in the form of, another product such as a cosmetic; a hair care or in particular a skin care preparation (for example a skin cleanser, toner or moisturiser, or a shampoo); a deodorant or anti-perspirant; a cosmetic (eg a makeup product); a cleansing preparation (for example a hand, body or facial wash); a pharmaceutical (which includes veterinary) preparation; a cosmeceutical preparation; a toiletry product (for instance a bath or shower additive or a soap); a laundry or other fabric treatment product; or an item of clothing or accessory therefor (for example a liner for a shoe). The formulation may be, or be incorporated into, a wash-off skin treatment product such as a skin cleanser, or in particular a leave-on skin treatment product.

A product as just described may be, according to the invention, for use in the treatment of either acne or body odour. The product may carry the antibacterial or anti-acne formulation. It may for instance be coated with the formulation, or it may be impregnated with the formulation, or the formulation may be contained in any location in or on the product.

A formulation for use according to the invention may be prepared in situ, at or immediately before its point of use, for instance its application to the skin or another surface. It may for instance be prepared from a kit which comprises sources of (a) tropone or a substituted tropone and (b) a metal or metal salt selected from copper, copper salts, bismuth, bismuth salts, silver, silver salts, and mixtures thereof, together with instructions for combining the two components so as to make the formulation at or before the point of intended use, and/or for the co-administration of the two components to a surface such as the skin. The two components may each be present in a suitable respective vehicle.

According to one embodiment, a formulation or kit for use according to the invention may contain both an optionally substituted tropone and an appropriate metal or metal salt, each encapsulated (for instance microencapsulated) in a separate delivery vehicle; this might for instance allow their release, and hence their contact with one another, only at the intended site of administration.

A formulation for use according to the invention may be marketed with an indication that it has antibacterial and/or anti-acne activity, or enhanced antibacterial and/or anti-acne activity. The marketing of such a formulation may include an activity selected from (a) enclosing the formulation in a container or package that comprises the relevant indication; (b) packaging the formulation with a package insert that comprises the indication; (c) providing the indication in a publication that describes the formulation; and (d) providing the indication in a commercial which is aired for instance on the radio, television or internet. The activity or enhancement may be attributed, in such an indication, at least partly to the presence of either or both of the tropone (a) and the metal or metal salt (b). The invention may involve assessing the activity of the formulation during or after its preparation, for instance against one or more of the pathogens referred to below. It may involve assessing the activity both before and after incorporation of the tropone (a) and/or the metal or metal salt (b), for example so as to confirm that either or both contribute to the antibacterial or anti-acne activity of the formulation.

An antibacterial or anti-acne formulation for use according to the invention may be prepared by mixing together (a) tropone or a substituted tropone and (b) a metal or metal salt selected from copper, copper salts, bismuth, bismuth salts, silver, silver salts, and mixtures thereof, suitably together with a pharmaceutically acceptable vehicle.

In a formulation for use according to the invention, the combination of the tropone (a) and the metal or metal salt (b) is typically present as an active (ie antibacterially active) agent. The combination may be present as an anti-acne agent (ie as an agent which is active against acne (which includes against a symptom and/or a cause of acne and/or against one or more micro-organisms associated with acne)). Suitably the two active agents are not used purely or even primarily as skin or hair conditioning agents, and/or as insecticides, and/or as anticancer agents, and/or as antidandruff agents, and/or as anti-Legionella agents, and/or for the inhibition of metalloproteinases, and/or for the promotion of hair growth, and/or as wound healing agents.

It is possible that the antibacterial and/or anti-acne activity of a combination of the optionally substituted tropone (a) with the metal or metal salt (b) may be at least partly due to the formation of a reaction product which itself has antibacterial and/or anti-acne activity. The invention may thus embrace an antibacterial or anti-acne formulation containing a reaction product formed between (a) tropone or a substituted tropone and (b) a metal or metal salt selected from copper, copper salts, bismuth, bismuth salts, silver, silver salts, and mixtures thereof, for use in the treatment of either acne or body odour; the reaction product may be formed in situ immediately prior to, or at the point of, use.

In the present context, antibacterial activity embraces activity against both Gram-positive and Gram-negative bacteria, in particular Gram-positive bacteria. It may be growth inhibitory activity or more preferably biocidal (ie lethal to the relevant organism). It may comprise activity against sessile and/or planktonic bacteria; in other words, it may comprise activity against bacteria which are growing as or in a biofilm. In the context of this invention, activity against a particular species of micro-organism may be taken to mean activity against at least one, or against two or more, strains of that species.

Antibacterial activity may be or include the ability to disrupt and/or suppress biofilm formation by the relevant organism. In the present context, the disruption of biofilm formation embraces any negative effect on the ability of a micro-organism to form, maintain or exist in a biofilm, and/or on a biofilm already formed by the organism. Thus, it may involve reducing the amount of a previously formed biofilm, and/or impairing such a biofilm. It may involve killing or inhibiting sessile micro-organisms within a biofilm.

Suppression of biofilm formation embraces any degree of impairment (including complete prevention) of the ability of a micro-organism to form, or more typically to co-aggregate with, a biofilm. It thus embraces total or partial impairment, including reducing the amount and/or strength of biofilm which the organism is able to form and/or the speed with which it is able to do so. It may involve preventing or reducing the growth or the rate of growth of an existing biofilm formed by the organism.

In an embodiment, a formulation for use according to the invention is active against one or more bacteria associated with acne, in particular propionibacteria such as P. acnes and in some instances P. granulosum. In an embodiment, it is active against one or more bacteria implicated in body odour, in particular in the axillae or feet, for example against one or more corynebacteria such as those identified below.

The formulation may be active against bacteria, in particular propionibacteria, which are wholly or partially resistant to one or more antibiotics, for instance those which are in common clinical use. For example it may be active against one or more macrolide-lincosamide-streptogramin (MLS) resistant and/or macrolide-lincosamide-streptogramin-ketolide (MLSK) resistant strains of bacteria. In particular it may be active against one or more erythromycin-resistant, clindamycin-resistant and/or tetracycline-resistant strains of bacteria, for example P. acnes strains, the term tetracycline here referring to the class of antibiotics including for example minocycline and doxycycline as well as the specific antibiotic known as tetracycline.

In an embodiment of the invention, the formulation is for use in the treatment of acne. In an embodiment, it is for use in the treatment of body odour.

In a further aspect, the formulation may be for use in the treatment of a condition which is caused, transmitted and/or exacerbated by (in particular either caused or transmitted by, more particularly caused by) propionibacteria. It may for instance be for use against an opportunistic infection which is caused, transmitted and/or exacerbated by propionibacteria, for instance an infection associated with an indwelling surgical device (a prosthetic joint, shunt, stent or catheter, for example). In the latter case, the device may be coated or impregnated with the formulation. The formulation may be for use in treating an infected wound, burn or ulcer. It may be for use against any other infection or condition which involves or can involve propionibacteria, for example an eye infection such as endophthalmitis.

In the context of the present invention, treatment of a condition encompasses both therapeutic and prophylactic treatment, in either a human or animal but in particular a human. It may involve complete or partial eradication of the condition, removal or amelioration of associated symptoms, arresting subsequent development of the condition, and/or prevention of, or reduction of risk of, subsequent occurrence of the condition. It will typically involve use of the tropone (a) and the metal or metal salt (b) as an antibacterial and/or anti-acne combination.

Treatment may involve use of the formulation against bacterial biofilm formation. The biofilm may be formed by, and/or may be associated with, a propionibacterium, in particular a cutaneous propionibacterium, or a coryneform bacterium.

In general the treatment may be administered orally, transdermally or topically. In an embodiment, it is administered locally. In an embodiment, it is administered topically.

In an embodiment of the invention, the formulation is for use against one or more propionibacteria, in particular against one or more cutaneous propionibacteria, more particularly against one or more bacteria associated with acne, such as P. acnes and in some instances P. granulosum.

Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment (which embraces prevention or reduction) of any of these symptoms, and references to use as an anti-acne agent may be construed accordingly. In particular, the treatment of acne encompasses the treatment (including prevention) of lesions and/or scarring associated with acne. It also encompasses the inhibition of propionibacterial activity which could cause or be otherwise associated with acne or its symptoms. In the context of the present invention, it may in particular be the treatment of inflamed acne lesions.

In general, the present invention will be used for the treatment of symptoms which are directly due to acne rather than for instance infections which may arise as a consequence of treating acne with other actives such as antibiotics, and/or secondary infections caused by opportunistic pathogens, which can arise in skin already affected by acne. It will not generally be used for the treatment of symptoms which are acne-like but which are not etiologically the same as acne, for instance skin rashes caused by treatment with other medicaments such as epidermal growth factor receptor (EGFR) inhibitors.

In an embodiment, the formulation is for use in the treatment (which includes prevention) of body odour, in particular human body odour, for example in the axillae or feet. It may thus be for use against one or more bacteria implicated in this condition, in particular aerobic diphtheroids of the genus Corynebacterium, and/or other members of the bacterial human skin microflora such as cutaneous propionibacteria or coagulase-negative staphylococci.

As described above, in an embodiment the formulation may be prepared in situ, at or immediately before the point of administration. The invention thus pertains to any use of (a) tropone or a substituted tropone and (b) a metal or metal salt selected from copper, copper salts, bismuth, bismuth salts, silver, silver salts, and mixtures thereof, in the treatment of either acne or body odour, the two components being administered either simultaneously or sequentially.

A second aspect of the invention provides the use of a formulation containing (a) tropone or a substituted tropone and (b) a metal or metal salt selected from copper, copper salts, bismuth, bismuth salts, silver, silver salts, and mixtures thereof, in the manufacture of a medicament (typically a formulation) for the treatment of either acne or body odour. The tropone (a) and the metal or metal salt (b) will typically be used as an antibacterial combination in the manufacture of the medicament, and/or as an anti-acne combination.

A third aspect provides a method of treatment of a human or animal patient suffering from or at risk of suffering from either acne or body odour, the method involving administering to the patient a therapeutically (which term includes prophylactically) effective amount of a formulation containing (a) tropone or a substituted tropone and (b) a metal or metal salt selected from copper, copper salts, bismuth, bismuth salts, silver, silver salts, and mixtures thereof. The formulation is suitably administered in an antibacterially effective amount. It may be administered by any appropriate route, suitably topically.

In an embodiment of the third aspect of the invention, the formulation is administered to a human patient. The patient is suitably suffering from the relevant condition.

The invention further provides, according to a fourth aspect, the use together of (a) tropone or a substituted tropone and (b) a metal or metal salt selected from copper, copper salts, bismuth, bismuth salts, silver, silver salts, and mixtures thereof, as a combined antibacterial agent, and/or as a combined anti-acne agent, for example in the manufacture of an antibacterial or anti-acne formulation.

A fifth aspect provides the use of a combination of (a) tropone or a substituted tropone and (b) a metal or metal salt selected from copper, copper salts, bismuth, bismuth salts, silver, silver salts, and mixtures thereof, for non-therapeutic purposes. In an embodiment of this fifth aspect, the formulation is used as an anti-acne or in particular a skin care agent for non-therapeutic purposes, for example for cosmetic purposes such as to improve the appearance, feel or smell of the skin. In an embodiment, it is used as an anti-body odour or in particular a deodorising agent for non-therapeutic purposes, for example for cosmetic purposes such as to improve the smell of a region of the body.

According to a sixth aspect, the invention provides a method for controlling the growth of a propionibacterium or a corynebacterium, the method comprising applying, to an area or surface which is infected or suspected to be infected or capable of becoming infected with the bacterium, a formulation containing (a) tropone or a substituted tropone and (b) a metal or metal salt selected from copper, copper salts, bismuth, bismuth salts, silver, silver salts, and mixtures thereof. Again the tropone (a) and the metal or metal salt (b) may be applied simultaneously or sequentially. They are suitably applied topically or locally. They may in particular be applied to an area or surface which is infected with the bacterium.

In accordance with the sixth aspect of the invention, “applying” a formulation to a surface embraces immersing the surface in the formulation.

The formulation may be applied to a non-living area or surface such as in a hospital or surgery. Thus the invention may be used to disinfect work surfaces, surgical or other instruments (including implants or prostheses) or other devices against bacteria such as propionibacteria or corynebacteria. It may be used to treat protective clothing such as surgical gloves, clothing or bedding. In an embodiment, it may be used to treat an implant or other device which is intended for use within the body.

“Controlling the growth” of a bacterium embraces inhibiting or preventing its growth, whether completely or partially, as well as killing either completely or partially a culture of the bacterium. It also embraces reducing the risk of subsequent growth of the bacterium in or on the area or surface being treated. It may embrace reducing the risk of transmission of the bacterium from the area or surface being treated to another area or surface and/or living body. The method of the invention may thus be used to treat an existing occurrence of the bacterium or to prevent a potential subsequent occurrence. Controlling the growth of a bacterium may also embrace the disruption and/or suppression of biofilm formation by the organism.

A seventh aspect of the invention provides the use of tropone or a substituted tropone, in an antibacterial or anti-acne formulation containing a metal or metal salt selected from copper, copper salts, bismuth, bismuth salts, silver, silver salts, and mixtures thereof, for the purpose of increasing the antibacterial and/or anti-acne activity of the formulation and/or of reducing the amount of the metal or metal salt in the formulation without or without undue loss of antibacterial or anti-acne activity.

An increase in antibacterial or anti-acne activity may be as compared to that of the metal or metal salt alone, at the same concentration as used when combined with the optionally substituted tropone. Ideally the increase is as compared to the sum of the activities of the tropone and the metal or metal salt individually, again at the same respective concentrations as used when the two are combined.

A reduction in the amount of the metal or metal salt in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the metal or metal salt; it may be manifested by a change in the properties of the formulation, for example by a change (including a reduction) in colour. According to the invention, the optionally substituted tropone may therefore be used for the dual purposes of reducing an undesired property of a formulation containing a metal or metal salt selected from copper, copper salts, bismuth, bismuth salts, silver, silver salts, and mixtures thereof, without or without undue loss of antibacterial or anti-acne activity.

The optionally substituted tropone may be used without any reduction in antibacterial or anti-acne activity compared to the level exhibited by the formulation prior to addition of the tropone. It may be used to give an increase in antibacterial or anti-acne activity, in particular in vivo. It may however be used to reduce the amount of the metal or metal salt present, and/or its associated side effects, and/or to alter a property of the formulation, whilst maintaining the antibacterial or anti-acne activity of the resultant formulation at a level, albeit lower than that which it would otherwise have exhibited, which is still acceptable in the context of its intended use.

An eighth aspect of the invention provides the use of a metal or metal salt selected from copper, copper salts, bismuth, bismuth salts, silver, silver salts, and mixtures thereof, in an antibacterial or anti-acne formulation containing tropone or a substituted tropone, for the purpose of increasing the antibacterial and/or anti-acne activity of the formulation and/or of reducing the amount of the optionally substituted tropone in the formulation without or without undue loss of antibacterial or anti-acne activity. The above comments regarding the seventh aspect of the invention apply mutatis mutandis to the eighth aspect.

Throughout the description and claims of this specification, the words “comprise” and “contain” and variations of the words, for example “comprising” and “comprises”, mean “including but not limited to”, and do not exclude other moieties, additives, components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Other features of the invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith. Moreover unless stated otherwise, any feature disclosed herein may be replaced by an alternative feature serving the same or a similar purpose.

Where upper and lower limits are quoted for a property, for example for the concentration of an active agent in a formulation, then a range of values defined by a combination of any of the upper limits with any of the lower limits may also be implied.

In this specification, references to properties such as solubilities, liquid phases and the like are—unless stated otherwise—to properties measured under ambient conditions, ie at atmospheric pressure and at a temperature of from 18 to 25° C., for example about 20° C.

The present invention will now be further described with reference to the following non-limiting examples and the accompanying figures, of which:

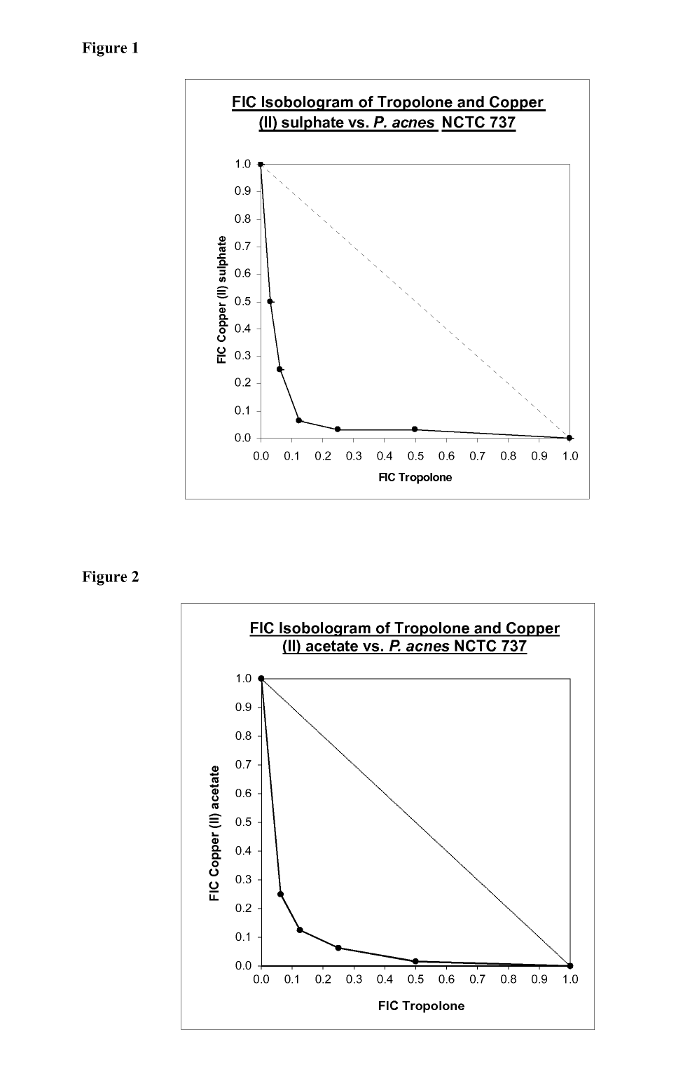

FIG. 1 is an isobologram showing FIC (fractional inhibitory concentration) values for mixtures of tropolone and copper (II) sulphate in assays against the bacterium P. acnes NCTC 737, as referred to in Example 2 below;

FIG. 2 is an isobologram showing FIC values for mixtures of tropolone and copper (II) acetate in assays against the bacterium P. acnes NCTC 737, as referred to in Example 6 below;

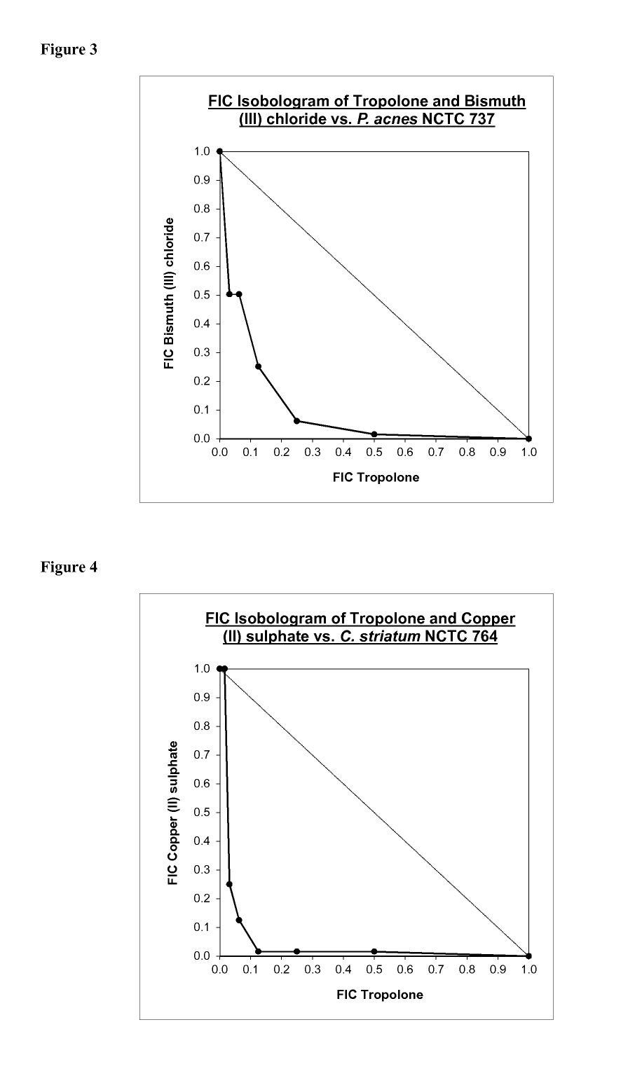

FIG. 3 is an isobologram showing FIC values for mixtures of tropolone and bismuth (III) chloride in assays against the bacterium P. acnes NCTC 737, as referred to in Example 8 below;

FIG. 4 is an isobologram showing FIC values for mixtures of tropolone and copper (II) sulphate in assays against the bacterium C. striatum NCTC 764, as referred to in Example 13 below;

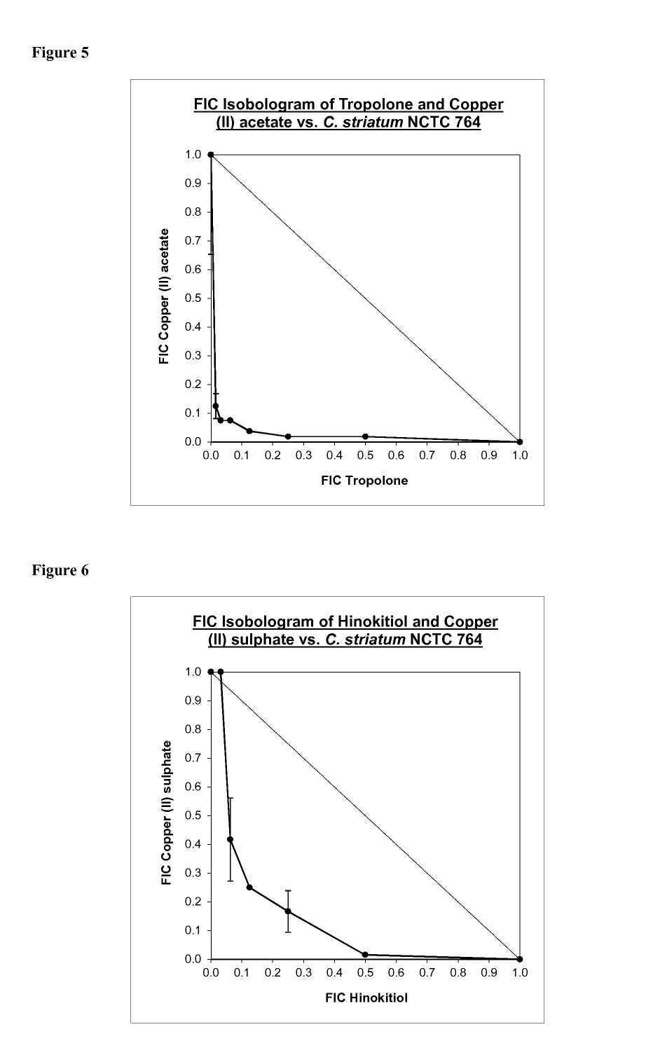

FIG. 5 is an isobologram showing FIC values for mixtures of tropolone and copper (II) acetate in assays against the bacterium C. striatum NCTC 764, as referred to in Example 13;

FIG. 6 is an isobologram showing FIC values for mixtures of hinokitiol and copper (II) sulphate in assays against the bacterium C. striatum NCTC 764, as referred to in Example 15 below; and

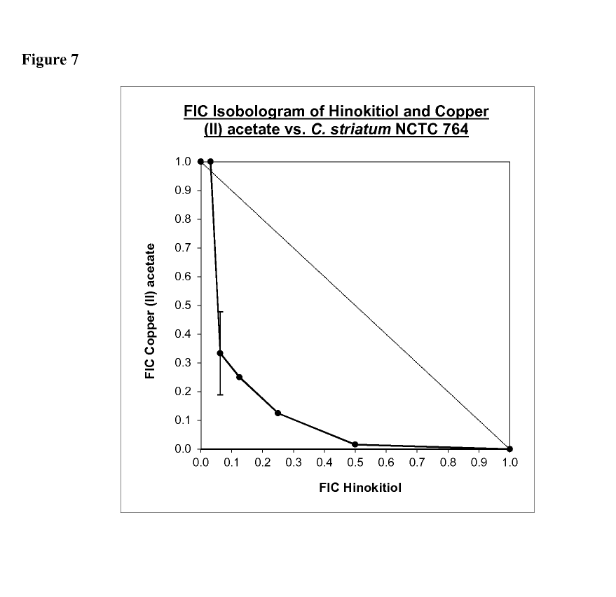

FIG. 7 is an isobologram showing FIC values for mixtures of hinokitiol and copper (II) acetate in assays against the bacterium C. striatum NCTC 764, as referred to in Example 15.

### DETAILED DESCRIPTION

Experimental tests were conducted to determine the antibacterial activity of formulations containing a substituted tropone and either a copper, a bismuth or a silver salt.

Test Micro-Organisms

The first test organism used was Propionibacterium acnes NCTC 737. This is a propionibacterial strain and is the type strain of the genus; it is fully susceptible to antibiotics. The propionibacteria are clinically significant due to their involvement in acne. They are also opportunistic pathogens in compromised hosts. Activity observed against these micro-organisms is expected to be a good predictor of activity against acne.

The propionibacteria were cultured and maintained on Wilkins-Chalgren Anaerobe Medium (agar and broth) at pH 6.0; all cultures were incubated anaerobically at 37° C. for 72 hours.

The second test organism used was Corynebacterium striatum NCTC 764. This is a Gram-positive bacterium which is closely related to organisms (aerobic diphtheroids of the genus Corynebacterium) that cause body odour (Guillet et al, Eur J Dermatol, 2000 Jan-Feb, 10(1): 41-2). The C. striatum was cultured and maintained on Mueller-Hinton Medium (agar and broth) at pH 7.3 (±0.1); cultures were incubated aerobically at 37° C. for 18-24 hours.

The following tests were carried out to assess antimicrobial activity against the test organisms.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μl of liquid per well. The wells contained liquid culture medium and ranges of decreasing concentrations of the relevant test compound in doubling dilutions (eg 1000, 500, 250, 125 . . . μg/ml, etc, down to 0.49 μg/ml). The culture media were as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, ie the lowest concentration for which the liquid in the well remained clear.

The assays included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Biocidal Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target microbial population to recover.

(c) Disc Diffusion Assay (DDA)

This is an internationally recognised standard method for qualitatively assessing the antimicrobial activity of a compound or combination of compounds.

A sterile paper disc was impregnated with a sample of the test compound(s) in a suitable solvent (or applied neat) and 30 minutes allowed for any of the solvents to evaporate (where possible). The disc was then placed on an agar plate onto which the test micro-organism had been inoculated. The plate was then incubated under the conditions described above, following which it was examined visually for signs of microbial growth. If the test compound(s) had antimicrobial activity, a circular zone of no growth would be obtained around the disc. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound(s), although other factors such as test compound mobility through the agar gel may also influence the result.

(d) Synergy Disc Diffusion Assay (SDDA)

This is a variation on the DDA method, in which two compounds are tested together for their combined antimicrobial activity.

Two test compounds A and B were placed on a single paper disc and the above described DDA procedure repeated. An increase in diameter of the zone of inhibition, compared to the greater of the zone diameters for the two compounds individually, was taken to indicate potential antimicrobial synergy. In practical terms, an increase of greater than 5 mm could be treated as significant.

(e) Fractional Inhibitory Concentration (FIC) Assay

This assay was used to determine the mode of interaction between two antimicrobial test compounds A and B. It was similar to the MIC assay, utilising a 96-well microtitre plate and liquid culture medium. The test compounds were added together to each well at a range of concentrations starting at their respective MIC values and descending in doubling dilutions as with the MIC assay. Typically an 8×8 array of wells could be used to combine 8 different concentrations of compound A (from its MIC downwards, including zero) with 8 different concentrations of compound B (from its MIC downwards, including zero).

The wells were inoculated with freshly grown micro-organism and incubated under the conditions described above.

As for the MIC assay, the results were read by the naked eye. A minimum inhibitory concentration was recorded for each combination of A and B. A fractional FIC index (FICI) was then calculated for each compound in that mixture, and these two indices were added together to give an overall FICI indicative of the mode of interaction.

Thus for each mixture tested, the FIC for compound A (FIC_{A})=MIC for (A+B)/MIC for A alone. Similarly the FIC for compound B (FIC_{B})=MIC for (A+B)/MIC for B alone. The overall FICI=FIC_{A}+FIC_{B}.

An FICI of 0.5 or less was taken to indicate synergy, a value greater than 0.5 to 4.0 an indifferent effect and values greater than 4.0 antagonism (ie the two compounds counter one another's activity, leading overall to a diminished antimicrobial effect) (see Odds F C, “Synergy, antagonism, and what the chequerboard puts between them”, J Antimicrob Chemother, 2003; 52:1). These results can be depicted visually on a plot (isobologram) of FIC_{A} against FIC_{B} for the mixtures tested.

(f) Synthetic Sebum Time-to-Kill Assay

This quantitative assay was designed to assess the level of kill of a P. acnes culture in a synthetic sebum (non-aqueous environment) over a defined time period (in this case 4 hours).

A culture of P. acnes NCTC 737 was inoculated into a synthetic sebum (a liquid mixture of lipid components designed to simulate human sebum) containing the relevant test compound dissolved in 1-octanol. From this culture, samples were taken after 4 hours, 10-fold serially diluted in wash fluid (0.075 M sodium phosphate buffer, pH 7.9, 0.1% v/v Triton-X 100) and inoculated onto agar plates (in triplicate). The plates were then incubated as described above and subsequently examined visually for growth. The numbers of viable microbial colonies on the plates were counted with the aid of a colony counter and converted to colony-forming units per ml (cfu/ml) using the appropriate dilution factor. Cell counts conducted in the absence of test compound acted as positive controls.

(g) Fixed Ratio Combination MIC/MBC Assays

This method used a sterile 96-well microtitre plate, capable of holding about 200 μl of liquid per well. The wells contained liquid culture medium and ranges of decreasing concentrations of the relevant test compounds alone in doubling dilutions (eg 1000, 500, 250, 125 . . . μg/ml, etc. down to 0.49 μg/ml) or doubling dilutions of a combination of two compounds mixed initially at a fixed concentration ratio (based on molarity in this instance). The culture medium was as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compounds alone or concentrations of the mixture of the two test compounds required to inhibit microbial growth, ie the liquid in the well remained clear.

The assays were conducted in triplicate and included both negative (culture medium with no micro-organisms) and positive (culture medium plus diluting solvent(s) plus micro-organism) controls.

Following the MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The minimum bactericidal concentration (MBC) was taken to be the lowest test compound concentration alone or the lowest concentrations of the mixture of the two test compounds for which the incubated sample showed no growth.

A fractional inhibitory concentration index (FICI) or fractional bactericidal concentration index (FBCI) was then calculated for each compound in that mixture, and these two indices were added together to give an overall FICI or FBCI to indicate of the mode of interaction.

Thus for each mixture tested, the FIC for compound A (FIC_{A})=MIC for (A+B)/MIC for A alone. Similarly the FIC for compound B (FIC_{B})=MIC for (A+B)/MIC for B alone. The overall FICI=FIC_{A}+FIC_{B}.

FBC & FBCI were calculated in the same manner using recorded MBC values.

An FICl/FBCI of 0.5 or less was taken to indicate synergy, a value greater than 0.5 to 4.0 an indifferent effect and values greater than 4.0 antagonism (ie the two compounds counter one another's activity, leading overall to a diminished antimicrobial effect) (see Odds F C, above).

### Example 1

### Activity Against P. acnes (Tropolone+Copper Sulphate)

The following experiments all used P. acnes NCTC 737 as the test organism.

MIC, MBC and DDA assays, as described above, were carried out using the test compounds tropolone and copper (II) sulphate pentahydrate (both sourced from Sigma-Aldrich, UK). The two compounds were then subjected in combination to the SDDA assay described above. Increases in zone diameter (mm) were measured with respect to those observed for the copper sulphate (which had showed larger zone diameters during the previous disc diffusion assays on the individual compounds).

For all the (S)DDA experiments, 200 μg of the tropolone and 12.5 μg of the copper sulphate were loaded onto each disc. The tropolone was dissolved in ethanol and the copper sulphate in deionised water. MIC/MBC experiments were performed in duplicate and (S)DDA experiments in triplicate. The results are shown in Table 1 below.

TABLE 1

| | | | Copper |
|---|---|---|---|
| | Copper | | sulphate + |
| Assay | sulphate | Tropolone | tropolone |
| MIC (μg/ml) | 15.6 | 15.6 | — |
| MBC (μg/ml) | 125 | 250 | — |
| MIC/MBC ratio | 0.125 | 0.06 | — |
| (S)DDA (mm) | 17.3 (±2.59) | 19.09 (±0.31) | 33.38 (±0.3) |
| SDDA increase (mm) | — | — | 14.29 |
| SDDA area increase (%) | — | — | 205.84 |

These data show that both the tropolone and the copper sulphate alone are active against P. acnes NCTC 737. When the tropolone and the copper salt are combined, however, the data demonstrate a synergistic antimicrobial interaction between the two, with a significant increase in zone diameter over that exhibited by either compound alone. This indicates the likely activity of the invented formulations as anti-acne agents, the propionibacteria being implicated in acne.

### Example 2

### Activity Against P. Acnes (FIC Assays) (Tropolone+Copper Sulphate)

Mixtures of tropolone and copper (II) sulphate, containing various relative proportions of the two actives, were subjected to FIC assays against P. acnes NCTC 737, as described above. The results were used to prepare FIC isobolograms. All assays were conducted in triplicate.

The overall FICI obtained for the mixtures was 0.19, representing the mean of three replicates. This indicates a synergistic interaction. A representative isobologram is shown in FIG. 1; the dashed line indicates where overall FICIs (ie FICₜᵣₒₚₒₗₒₙₑ+FIC_{copper (II) sulphate}) equal 1, which would indicate a purely indifferent interaction. FIG. 1 clearly demonstrates the synergistic activity of the combination of the tropolone and the copper salt against P. acnes NCTC 737.

### Example 3

### Activity Against P. Acnes (Synthetic Sebum Time-to-Kill Assays) (Tropolone+Copper Sulphate)

Mixtures of tropolone and copper (II) sulphate, containing various relative proportions of the two actives, were subjected to synthetic sebum time-to-kill assays against P. acnes NCTC 737, as described above. All assays were conducted in triplicate.

The results, including for tests on the individual test compounds, are shown in Table 2 below.

TABLE 2

| | Log10 cfu/ml | Log10 cfu/ml | Log10 |
|---|---|---|---|
| Test | (T0 hours) | (T4 hours) | change |
| Control (+) | 8.11 | 8.2 | 0.09 |
| Copper sulphate (97.5 mg/L) | 8.11 | 6.58 | −1.53 |
| Tropolone (195 mg/L) | 8.11 | 7.8 | −0.31 |
| Copper sulphate (97.5 | 8.11 | 3.0* | −5.11 |
| mg/L) + tropolone (195 | | | |
| mg/L) | | | |

*Lowest detectable level = 3.0 Log10 cfu/ml

At the concentrations tested both the tropolone and in particular the copper sulphate demonstrated some level of antimicrobial activity against P. acnes NCTC 737 in a lipid environment. However, when the tropolone and the copper salt were combined a synergistic effect was observed, as they reduced P. acnes viability below the lowest detectable level (a >99.999% reduction). This retention of activity in a lipid environment—which mimics that of the human skin—further indicates the utility of the invented formulations as topical anti-acne agents.

### Example 4

### Activity Against P. Acnes (Tropolone+Other Copper Salts)

A series of copper salts was tested with tropolone against P. acnes NCTC 737 using (S)DDA tests as described in Example 1. The MIC and MBC values for each copper salt were also measured as in Example 1.

For the (S)DDA experiments, 12.5 μg of each test compound was loaded onto each disc, with the exception of copper (II) gluconate and copper (II) picolinate which were loaded at 25 μg, copper (II) silicate at 12.4 μg and sodium copper (II) chlorophyllin at 200 μg. The copper salts were dissolved in deionised water with the exception of copper (II) carbonate basic, which was dissolved in ethanol; and copper (I) iodide, copper (II) salicylate, copper (II) usnate, copper (II) citrate dihydrate and copper (II) picolinate, all of which were dissolved in DMSO. The tropolone was dissolved in ethanol.

All compounds were sourced from Sigma-Aldrich, UK with the exception of copper (II) salicylate (made in-house), copper (II) silicate (Convé Ltd, UK), copper (II) PCA (Aston Chemicals Ltd, UK), copper (II) usnate (Just For Today Ltd, UK), copper (II) citrate dihydrate (Chemos GmBH, Germany), copper (II) acetylmethionate (Exsymol SAM, Monaco) and copper (II) picolinate (Carbone Scientific Co Ltd, UK).

MIC and MBC experiments were conducted in triplicate with the exception of those using copper (I) acetate, copper (I) iodide, copper (II) acetate, copper (II) gluconate, copper (II) salicylate and copper (II) silicate, which were performed in duplicate. (S)DDA experiments were performed in triplicate.

The MIC and MBC results are shown in Table 3 below and the (S)DDA results in Table 4. All results are collated from a number of experiments.

TABLE 3

| | MIC | MBC | MIC/MBC |
|---|---|---|---|
| Copper salt | (μg/ml) | (μg/ml) | ratio |
| Copper (I) acetate | 7.8 | 31.25 | 0.25 |
| Copper (I) iodide | 7.8 | 31.25 | 0.25 |
| Copper (II) acetate | 3.9 | 125 | 0.03 |
| Copper (II) acetylmethionate | 7.8 | 125 | 0.06 |
| Copper (II) carbonate basic | 7.8 | 31.25 | 0.25 |
| Copper (II) chloride | 1.95 | 31.25 | 0.06 |
| Copper (II) citrate dihydrate | 1.95 | 31.25 | 0.06 |
| Copper (II) gluconate | 62.5 | 125 | 0.5 |
| Copper (II) PCA | 15.6 | 62.5 | 0.25 |
| Copper (II) picolinate | 62.5 | 62.5 | 1 |
| Copper (II) salicylate | 7.8 | 31.25 | 0.25 |
| Copper (II) silicate | 4.84 | 38.75 | 0.125 |
| Copper (II) usnate | 0.25 | 1.95 | 0.125 |
| EDTA copper (II) disodium salt | 7.8 | >250 | <0.03 |
| Sodium copper (II) chlorophyllin | 15.6 | 31.25 | 0.5 |

TABLE 4

| | | (S)DDA | | SDDA |
|---|---|---|---|---|
| | | with | SDDA | area |
| | *DDA | tropolone | increase | increase |
| Copper salt | (mm) | (mm) | (mm) | (%) |
| Copper (I) acetate | 32.14 | 32.35 | 0.21 | 1.29 |
| | (±0.78) | (±1.47) | | |
| Copper (I) iodide | 29.66 | 33.07 | 3.41 | 24.28 |
| | (±2.35) | (±0.95) | | |
| Copper (II) acetate | 25.63 | 30.80 | 5.17 | 44.39 |
| | (±0.65) | (±0.72) | | |
| Copper (II) acetylmethionate | 10.23 | 26.67 | 13.44 | 306.27 |
| | (±0.31) | (±0.54) | | |
| Copper (II) carbonate basic | 9.10 | 18.81 | 5.58 | 102.17 |
| | (±0.18) | (±5.2) | | |
| Copper (II) chloride | 32.14 | 32.45 | 0.31 | 1.94 |
| | (±0.90) | (±1.0) | | |
| Copper (II) citrate dihydrate | 32.04 | 32.45 | 0.41 | 2.60 |
| | (±1.76) | (±0.72) | | |
| Copper (II) gluconate | 26.25 | 31.11 | 4.86 | 40.43 |
| | (±1.40) | (±1.09) | | |
| Copper (II) PCA | 23.36 | 29.15 | 5.79 | 55.70 |
| | (±0.72) | (±0.62) | | |
| Copper (II) picolinate | 35.25 | 32.66 | −2.58 | −14.13 |
| | (±1.56) | (±0.65) | | |
| Copper (II) salicylate | 28.32 | 32.87 | 4.55 | 34.70 |
| | (±0.95) | (±0.31) | | |
| Copper (II) silicate | 33.80 | 34.32 | 0.52 | 3.08 |
| | (±0.82) | (±0.95) | | |
| Copper (II) usnate | 38.35 | 42.38 | 4.03 | 22.13 |
| | (±0.78) | (±1.25) | | |
| EDTA copper (II) disodium | 10.03 | 27.39 | 14.16 | 328.62 |
| salt | (±0.18) | (±0.65) | | |
| Sodium copper (II) chlorophyllin | 0.00 | 23.67 | 10.44 | 220.07 |
| | (±0.0) | (±0.78) | | |

*The DDA for tropolone alone was recorded at 13.23 mm (±0.65 mm).

These data show that combinations of tropolone with a range of different copper salts are antibacterially active against P. acnes NCTC 737, and thus likely to be of use as anti-acne agents. In most cases the data indicate a synergistic interaction between the two actives, often with a significant increase in zone diameter over that exhibited by either compound alone.

### Example 5

### Activity Against P. Acnes (Fixed Ratio Combination MIC/MBC Assays) (Tropolone+Copper Salts)

Fixed ratio combination MIC/MBC assays, as described above, were carried out using the test compounds tropolone, copper (II) acetate and copper (II) sulphate. The tropolone was dissolved in ethanol and the copper salts in deionised water. All experiments were performed in triplicate. The results are shown in Tables 5 and 6 below, for copper (II) acetate and copper (II) sulphate respectively.

TABLE 5

| | Molecular | MIC | | MBC | |
|---|---|---|---|---|---|
| Compound | ratio | (μg/ml) | FICI | (μg/ml) | FBCI |
| Tropolone (Trp) | | 31.25 | | >62.5* | |
| Copper (II) | | 11.5 | | 46 | |
| acetate (CuA) | | | | | |
| Trp + CuA | 2:1 | 3.9/2.9 | 0.38 | 7.8/5.8 | 0.188^{‡} |

*62.5 μg/ml was the highest concentration tested;
^{‡}Based on the MBC of tropolone being 125 μg/ml.

TABLE 6

| | Molecular | MIC | | MBC | |
|---|---|---|---|---|---|
| Compound | ratio | (μg/ml) | FICI | (μg/ml) | FBCI |
| Tropolone (Trp) | | 31.25 | | >62.5* | |
| Copper (II) | | 20.4 | | >40.9** | |
| sulphate (CuS) | | | | | |
| Trp + CuS | 1:1 | 7.8/10.5 | 0.75 | 31.25/20.4 | 0.5^{‡} |

*62.5 μg/ml was the highest concentration tested;
**40.9 μg/ml was the highest concentration tested;
^{†}Based on the MBC of CuS being 81.8 μg/ml.
^{‡}Based on the MBC of tropolone being 125 μg/ml.

Tables 5 and 6 show that when the tropolone is combined with either of the copper salts, a synergistic (FICI and FBCI recorded at ≦0.5, with the exception of the FICI value for copper (II) sulphate) antimicrobial interaction results between the two. This further indicates the likely utility of a formulation containing tropolone and a copper salt, as an anti-acne agent.

### Example 6

### Activity Against P. Acnes (FIC Assays) (Tropolone+Copper Acetate)

Mixtures of tropolone and copper (II) acetate, containing various relative proportions of the two actives, were subjected to FIC assays against P. acnes NCTC 737, as described above. The results were used to prepare FIC isobolograms. All assays were conducted in triplicate.

The overall FICI obtained for the mixtures was 0.25, representing the mean of three replicates. This indicates a synergistic interaction. A representative isobologram is shown in FIG. 2; the dashed line indicates where overall FICIs (ie FIC_{Tropolone}+FIC_{copper (II) acetate}) equal 1, which would indicate a purely indifferent interaction. FIG. 2 clearly demonstrates the synergistic activity of the combination of the tropolone and the copper salt against P. acnes NCTC 737.

### Example 7

### Activity Against P. Acnes (Tropolone+Bismuth Salts)

The following experiments used P. acnes NCTC 737 as the test organism.

Fixed ratio combination MIC/MBC assays, as described above, were carried out using as the test compounds (a) tropolone and (b) a series of bismuth salts (all sourced from Sigma-Aldrich, UK). The tropolone was dissolved in ethanol and the bismuth salts in DMSO. All experiments were performed in triplicate. The results are shown in Tables 7 to 11 below.

TABLE 7

| | Molecular | MIC | | MBC | |
|---|---|---|---|---|---|
| Compound | ratio | (μg/ml) | FICI | (μg/ml) | FBCI |
| Tropolone (Trp) | | 31.25 | | >62.5* | |
| Bismuth (III) | | 3.38 | | 27 | |
| chloride (BisC) | | | | | |
| Trp + BisC | 3:1 | 0.98/0.84 | 0.28 | 1.95/1.69 | 0.08^{‡} |

*62.5 μg/ml was the highest concentration tested;
^{‡}Based on the MBC of tropolone being 125 μg/ml.

TABLE 8

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Bismuth (III) | | 2.1 | | 8.5 | |
| citrate (BisCt) | | | | | |
| Trp + BisCt | 3:1 | 1.95/2.1 | 1.03 | 1.95/2.1 | 0.27^{‡} |

*62.5 μg/ml was the highest concentration tested;
^{‡}Based on the MBC of tropolone being 125 μg/ml.

TABLE 9

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Bismuth (III) | | >67.2** | | >67.2** | |
| subgallate (BisSg) | | | | | |
| Trp + BisSg | 3:1 | 1.95/2.1 | 0.05^{†} | 3.9/4.2 | 0.06^{†‡} |

*62.5 μg/ml was the highest concentration tested;
**67.2 μg/ml was the highest concentration tested;
^{†}Based on the MIC/MBC of BisSg being 134.4 μg/ml.
^{‡}Based on the MBC of tropolone being 125 μg/ml.

TABLE 10

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Bismuth (III) | | 3.9 | | 15.6 | |
| subnitrate (BisSn) | | | | | |
| Trp + BisSn | 15:1 | 0.49/1.95 | 0.51 | 0.98/3.9 | 0.26^{‡} |

*62.5 μg/ml was the highest concentration tested;
^{‡}Based on the MBC of tropolone being 125 μg/ml.

TABLE 11

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Bismuth (III) | | 11.6 | | >46.4** | |
| acetate (BisA) | | | | | |
| Trp + BisA | 3:1 | 3.9/2.9 | 0.31 | 7.8/5.8 | 0.13^{†‡} |

*62.5 μg/ml was the highest concentration tested;
**46.4 μg/ml was the highest concentration tested;
^{†}Based on the MBC of BisA being 92.8 μg/ml.
^{‡}Based on the MBC of tropolone being 125 μg/ml.

These data show that both the tropolone and most of the bismuth salts are active against P. acnes NCTC 737. When the tropolone is combined with a bismuth salt, the data demonstrate a synergistic (FICI and FBCI recorded at ≦0.5, with the exception of FICI values for bismuth (III) citrate and bismuth (III) subnitrate) antimicrobial interaction between the two. This indicates the likely utility of a formulation containing both tropolone and a bismuth salt, as an anti-acne agent.

### Example 8

### Activity Against P. Acnes (FIC Assays) (Tropolone+Bismuth Chloride)

Mixtures of tropolone and bismuth (III) chloride, containing various relative proportions of the two actives, were subjected to FIC assays against P. acnes NCTC 737, as described above. The results were used to prepare FIC isobolograms. All assays were conducted in triplicate.

The overall FICI obtained for the mixtures was 0.31, representing the mean of three replicates. This indicates a synergistic interaction. A representative isobologram is shown in FIG. 3; the dashed line indicates where overall FICIs (ie FIC_{Tropolone}+FIC_{Bismuth (III) chloride}) equal 1, which would indicate a purely indifferent interaction. FIG. 3 clearly demonstrates the synergistic activity of the combination of the tropolone and the bismuth salt against P. acnes NCTC 737.

### Example 9

### Activity Against P. Acnes (Hinokitiol+Copper & Bismuth Salts)

The following experiments all used P. acnes NCTC 737 as the test organism.

Fixed ratio combination MIC/MBC assays, as described above, were carried out using the test compounds hinokitiol, copper (II) acetate, copper (I) acetate, copper (II) sulphate and bismuth (III) chloride (all sourced from Sigma-Aldrich, UK). The hinokitiol and bismuth salt were dissolved in DMSO and the copper salts in deionised water. All experiments were performed in triplicate. The results are shown in Tables 12 to 15 below.

TABLE 12

| | Molecular | MIC | | MBC | |
|---|---|---|---|---|---|
| Compound | ratio | (μg/ml) | FICI | (μg/ml) | FBCI |
| Hinokitiol (Hin) | | 31.25 | | 62.5 | |
| Copper (II) | | 30.4 | | 60.8 | |
| sulphate (CuS) | | | | | |
| Hin + CuS | 2:1 | 15.6/7.6 | 0.75 | 15.6/7.6 | 0.38 |

TABLE 13

| | Molecular | MIC | | MBC | |
|---|---|---|---|---|---|
| Compound | ratio | (μg/ml) | FICI | (μg/ml) | FBCI |
| Hinokitiol (Hin) | | 31.25 | | 62.5 | |
| Copper (II) | | 34.6 | | 34.6 | |
| acetate (CuA) | | | | | |
| Hin + CuA | 2:1 | 7.8/4.3 | 0.38 | 15.6/8.6 | 0.5 |

TABLE 14

| | Molecular | MIC | | MBC | |
|---|---|---|---|---|---|
| Compound | ratio | (μg/ml) | FICI | (μg/ml) | FBCI |
| Hinokitiol (Hin) | | 31.25 | | 62.5 | |
| Copper (I) | | 23.3 | | 23.3 | |
| acetate (CuAc) | | | | | |
| Hin + CuAc | 1:1 | 7.8/5.8 | 0.5 | 15.6/11.7 | 0.75 |

TABLE 15

| | Molecular | MIC | | MBC | |
|---|---|---|---|---|---|
| Compound | ratio | (μg/ml) | FICI | (μg/ml) | FBCI |
| Hinokitiol | | 31.25 | | 62.5 | |
| (Hin) | | | | | |
| Bismuth (III) | | 10 | | 40 | |
| chloride | | | | | |
| (BisC) | | | | | |
| Hin + BisC | 3:1 | <0.98/<0.63 | 0.05^{†‡} | <0.98/<0.63 | 0.016^{†‡} |

^{†}Based on the MIC/MBC of hinokitiol in combination with bismuth chloride being 0.49 μg/ml
^{‡}Based on the MIC/MBC of bismuth chloride in combination with hinokitiol being 0.31 μg/ml.

Surprisingly, when the hinokitiol is combined with either a copper or a bismuth salt, at various molecular ratios, the data demonstrate a synergistic (FICI & FBCI recorded at ≦0.5, with the exception of the FICI for copper (II) sulphate and the FBCI of the copper (I) acetate) antimicrobial interaction between the two. This indicates the likely utility of a formulation containing both hinokitiol and either a copper or a bismuth salt, as an anti-acne agent.

### Example 10

### Topical Anti-Acne Formulations

The results from Examples 1 to 9 show that the combination of a substituted tropone and a copper or bismuth salt can be an effective antibacterial agent against the bacteria associated with acne. This can be of use in preparing antibacterial formulations, in particular for topical application to the skin, for either prophylactic or therapeutic use in any context where such bacteria are thought to be involved as possible sources of infection. More specifically, it can be of use in preparing anti-acne formulations, again suitably for topical use.

A topical formulation for use in treating acne may for example be prepared by combining a substituted tropone such as tropolone or hinokitiol with either copper or a copper salt such as copper sulphate or copper PCA, or with either bismuth or a bismuth salt such as bismuth subnitrate or bismuth gallate, in a suitable fluid vehicle and optionally together with conventional additives. Such vehicles and additives may be for instance as found in Williams' Transdermal and Topical Drug Delivery, Pharmaceutical Press, 2003 and other similar reference books, and/or in Rolland A et al, “Site-specific drug delivery to pilosebaceous structures using polymeric microspheres”, Pharm Res 1993; 10: 1738-44; Mordon S et al, “Site-specific methylene blue delivery to pilosebaceous structures using highly porous nylon microspheres: an experimental evaluation”, Lasers Surg Med 2003; 33: 119-25; and Alvarez-Roman R et al, “Skin penetration and distribution of polymeric nanoparticles”, J Controlled Release 2004; 99: 53-62.

The formulation may be prepared and administered using known techniques. It may for example take the form of a cream, lotion or in particular a gel.

The concentrations of the two active agents may be in the ranges described above, and will be determined based on the intended use of the formulation, its intended mode of administration and the activities of the particular chosen active agents. Suitably, the formulation is administered topically to acne-affected skin.

### Example 11

### Activity Against C. Striatum (Tropolone+Copper Sulphate)

The following experiment used C. striatum NCTC 764 as the test organism. Fixed ratio combination MIC/MBC assays, as described above, were carried out using the test compounds tropolone and copper (II) sulphate (both sourced from Sigma-Aldrich, UK). The tropolone was dissolved in ethanol and the copper sulphate in deionised water. All experiments were performed in triplicate. The results are shown in Table 16 below.

TABLE 16

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >40.9** | | >40.9** | |
| sulphate (CuS) | | | | | |
| Trp + CuS | 2:1 | 15.6/10.2 | 0.375^{†} | 15.6/10.2 | 0.25^{†‡} |

*62.5 μg/ml was the highest concentration tested;
**40.9 μg/ml was the highest concentration tested;
^{†}Based on the MIC/MBC of CuS being 81.8 μg/ml.
^{‡}Based on the MBC of tropolone being 125 μg/ml.

These data show that the tropolone alone is active against C. striatum NCTC 764. Surprisingly, when the tropolone and the copper salt are combined, the data demonstrate a synergistic (FICI & FBCI recorded at ≦0.5) antimicrobial interaction between the two. The presence of very low levels of copper significantly decreased both the MIC and MBC of tropolone when compared to those recorded for the compound alone. This indicates the likely activity of the invented formulations as agents against body odour, a condition in which the corynebacteria are implicated.

### Example 12

### Activity Against C. Striatum (Tropolone+Other Copper Salts)

The following experiments all used C. striatum NCTC 764 as the test organism.

Fixed ratio combination MIC/MBC assays, as described above, were carried out using the test compounds tropolone, copper (I) iodide, copper (II) acetate, copper (I) acetate, copper (II) D-gluconate, copper (II) salicylate, copper (II) silicate, copper (II) PCA, copper (II) usnate, copper (II) chloride, copper (II) citrate dihydrate, copper (II) glycinate, sodium copper (II) chlorophyllin, copper (II) carbonate and copper (II) picolinate (all sourced from Sigma-Aldrich, UK).

The tropolone was dissolved in ethanol and the copper salts in deionised water, with the exception of copper (I) iodide, copper (II) salicylate, copper (II) usnate, copper (II) citrate dehydrate and copper (II) picolinate which were dissolved in DMSO and copper (II) carbonate which was dissolved in ethanol. All experiments were performed in triplicate.

The results are shown in Tables 17 to 30 below. Data are collated from a number of experiments.

TABLE 17

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (I) | | >97.5** | | >97.5** | |
| iodide (CuI) | | | | | |
| Trp + CuI | 1:1 | 15.6/24.4 | 0.375^{†} | 15.6/24.4 | 0.25^{†‡} |

*62.5 μg/ml was the highest concentration tested;
**97.5 μg/ml was the highest concentration tested;
^{†}Based on the MIC/MBC of CuI being 195 μg/ml.
^{‡}Based on the MBC of tropolone being 125 μg/ml.

TABLE 18

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >46.5** | | >46.5** | |
| acetate (CuA) | | | | | |
| Trp + CuA | 2:1 | 15.6/11.6 | 0.375^{†} | 15.6/11.6 | 0.25^{†‡} |

*62.5 μg/ml was the highest concentration tested;
**46.5 μg/ml was the highest concentration tested;
^{†}Based on the MIC/MBC of CuA being 93 μg/ml.
^{‡}Based on the MBC of tropolone being 125 μg/ml.

TABLE 19

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (I) | | >62.8** | | >62.8** | |
| acetate (CuAc) | | | | | |
| Trp + CuAc | 1:1 | 7.8/7.8 | 0.188^{†} | 7.8/7.8 | 0.125^{†‡} |

*62.5 μg/ml was the highest concentration tested;
**62.8 μg/ml was the highest concentration tested;
^{†}Based on the MIC/MBC of CuAc being 125.6 μg/ml.
^{‡}Based on the MBC of tropolone being 125 μg/ml.

TABLE 20

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) D- | | >116** | | >116** | |
| gluconate (CuG) | | | | | |
| Trp + CuG | 2:1 | 15.6/29 | 0.375^{†} | 15.6/29 | 0.25^{†‡} |

*62.5 μg/ml was the highest concentration tested;
**116 μg/ml was the highest concentration tested;
^{†}Based on the MIC/MBC of CuG being 232 μg/ml.
^{‡}Based on the MBC of tropolone being 125 μg/ml.

TABLE 21

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >86** | | >86** | |
| salicylate (CuSl) | | | | | |
| Trp + CuSl | 2:1 | 7.8/11 | 0.188^{†} | 7.8/11 | 0.125^{†‡} |

*62.5 μg/ml was the highest concentration tested;
**86 μg/ml was the highest concentration tested;
^{†}Based on the MIC/MBC of CuSl being 172 μg/ml.
^{‡}Based on the MBC of tropolone being 125 μg/ml.

TABLE 22

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >35.7** | | >35.7** | |
| silicate (CuSil) | | | | | |
| Trp + CuSil | 2:1 | 7.8/4.5 | 0.188^{†} | 15.6/8.9 | 0.25^{†‡} |

*62.5 μg/ml was the highest concentration tested;
**35.7 μg/ml was the highest concentration tested;
^{†}Based on the MIC/MBC of CuSil being 71.4 μg/ml.
^{‡}Based on the MBC of tropolone being 125 μg/ml.

TABLE 23

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >81.9** | | >81.9** | |
| PCA (CuPCA) | | | | | |
| Trp + CuPCA | 2:1 | 7.8/10.2 | 0.188^{†} | 15.6/20.5 | 0.25^{†‡} |

*62.5 μg/ml was the highest concentration tested;
**81.9 μg/ml was the highest concentration tested;
^{†}Based on the MIC/MBC of CuPCA being 163.8 μg/ml.
^{‡}Based on the MBC of tropolone being 125 μg/ml.

TABLE 24

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | 3 | | 6 | |
| usnate (CuU) | | | | | |
| Trp + CuU | 2:1 | 1.95/6 | 2.03 | 7.8/24 | 4.06^{‡} |

*62.5 μg/ml was the highest concentration tested;
^{‡}Based on the MBC of tropolone being 125 μg/ml.

TABLE 25

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >34.4** | | >34.4** | |
| chloride (CuCl) | | | | | |
| Trp + CuCl | 2:1 | 15.6/8.6 | 0.375^{†} | 15.6/8.6 | 0.25^{†‡} |

*62.5 μg/ml was the highest concentration tested;
**34.4 μg/ml was the highest concentration tested;
^{†}Based on the MIC/MBC of CuCl being 68.8 μg/ml.
^{‡}Based on the MBC of tropolone being 125 μg/ml.

TABLE 26

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >45** | | >45** | |
| citrate hydrate | | | | | |
| (CuCit) | | | | | |
| Trp + CuCit | 2:1 | 15.6/11.25 | 0.375^{†} | 15.6/11.25 | 0.25^{†‡} |

*62.5 μg/ml was the highest concentration tested;
**45 μg/ml was the highest concentration tested;
^{†}Based on the MIC/MBC of CuCit being 90 μg/ml.
^{‡}Based on the MBC of tropolone being 125 μg/ml.

TABLE 27

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >54.2** | | >54.2** | |
| glycinate (CuGly) | | | | | |
| Trp + CuGly | 2:1 | 31.25/27.1 | 0.75^{†} | 31.25/27.1 | 0.5^{†‡} |

*62.5 μg/ml was the highest concentration tested;
**54.2 μg/ml was the highest concentration tested;
^{†}Based on the MIC/MBC of CuGly being 108.4 μg/ml.
^{‡}Based on the MBC of tropolone being 125 μg/ml.

TABLE 28

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Sodium copper | | 23.2 | | >185** | |
| (II) chlorophyllin | | | | | |
| (SCuC) | | | | | |
| Trp + SCuC | 2:1 | 7.8/23.2 | 1.125 | >62.5/>185 | n/a |

*62.5 μg/ml was the highest concentration tested;
**185 μg/ml was the highest concentration tested.

TABLE 29

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >28.3** | | >28.3** | |
| carbonate (CuCb) | | | | | |
| Trp + CuCb | 4:1 | 15.6/7.1 | 0.375^{†} | 15.6/7.1 | 0.25^{†‡} |

*62.5 μg/ml was the highest concentration tested;
**28.3 μg/ml was the highest concentration tested;
^{†}Based on the MIC/MBC of CuCb being 56.6 μg/ml.
^{‡}Based on the MBC of tropolone being 125 μg/ml.

TABLE 30

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Copper (II) | | >78.8** | | >78.8** | |
| picolinate (CuP) | | | | | |
| Trp + CuP | 2:1 | 7.8/9.8 | 0.188^{†} | 31.25/39.4 | 0.5^{†‡} |

*62.5 μg/ml was the highest concentration tested;
**78.8 μg/ml was the highest concentration tested;
^{†}Based on the MIC/MBC of CuP being 157.6 μg/ml.
^{‡}Based on the MBC of tropolone being 125 μg/ml.

Tables 17 to 30 show that, although many of the copper salts are not themselves highly active against the test organism, they are able to potentiate the activity of the tropolone. Surprisingly, when the tropolone and the copper salts are combined at various molecular ratios, the data demonstrate a synergistic (FICI & FBCI recorded at ≦0.5) antimicrobial interaction (with the exception of copper (II) usnate, sodium copper (II) chlorophyllin and copper (II) glycinate, the latter showing synergy in its FBCI only) between the two. The presence of very low levels of copper significantly decreases both the MIC and the MBC of tropolone when compared to those recorded for the compound alone. This further indicates the likely activity of the invented formulations as anti-body odour agents.

### Example 13

### Activity Against C. Striatum (FIC Assays) (Tropolone+Copper Sulphate/Acetate)

Mixtures of tropolone and either copper (II) sulphate or copper (II) acetate, containing various relative proportions of the two actives, were then subjected to FIC assays against C. striatum NCTC 764, as described above. The results were used to prepare FIC isobolograms. All assays were conducted in triplicate.

Representative isobolograms are shown in FIGS. 4 and 5, for copper (II) sulphate and copper (II) acetate respectively. The dashed lines indicate in each case where overall FICIs (ie FIC_{Tropolone}+FIC_{Copper (II) sulphate} or FIC_{Tropolone}+FIC_{Copper (II) acetate}) equal 1, which would indicate a purely indifferent interaction. FIGS. 4 and 5 clearly demonstrate the synergistic (FICI=0.14 & 0.11) activity of the combination of tropolone with either copper (II) sulphate or copper (II) acetate against C. striatum NCTC 764. This in turn indicates good anti-body odour activity for the invented combinations.

### Example 14

### Activity Against C. Striatum (Tropolone+Bismuth & Silver Salts)

The following experiments used C. striatum NCTC 764 as the test organism. Fixed ratio combination MIC/MBC assays, as described above, were carried out using the test compounds tropolone, bismuth (III) citrate, bismuth (III) subgallate, bismuth (III) subnitrate and silver (I) citrate hydrate (all sourced from Sigma-Aldrich, UK). The tropolone and silver (I) citrate hydrate were dissolved in ethanol, and the bismuth salts in DMSO. All experiments were performed in triplicate.

The results are shown in Tables 31 to 34 below. Data are collated from a number of experiments.

TABLE 31

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Bismuth (III) | | >67.9** | | >67.9** | |
| citrate (BisC) | | | | | |
| Trp + BisC | 3:1 | 7.8/8.5 | 0.188^{†} | 7.8/8.5 | 0.125^{†‡} |

*62.5 μg/ml was the highest concentration tested;
**67.9 μg/ml was the highest concentration tested;
^{†}Based on the MIC/MBC of BisC being 135.8 μg/ml.
^{‡}Based on the MBC of tropolone being 125 μg/ml.

TABLE 32

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Bismuth (III) | | >67.2** | | >67.2** | |
| subgallate (BisSg) | | | | | |
| Trp + BisSg | 3:1 | 7.8/8.4 | 0.188^{†} | 7.8/8.4 | 0.125^{†‡} |

*62.5 μg/ml was the highest concentration tested;
**67.2 μg/ml was the highest concentration tested;
^{†}Based on the MIC/MBC of BisSg being 134.4 μg/ml.
^{‡}Based on the MBC of tropolone being 125 μg/ml.

TABLE 33

| | Molecular | MIC | | MBC | |
|---|---|---|---|---|---|
| Compound | ratio | (μg/ml) | FICI | (μg/ml) | FBCI |
| Tropolone (Trp) | | 62.5 | | >62.5* | |
| Bismuth (III) | | >249.4** | | >249.4** | |
| subnitrate (BisSn) | | | | | |
| Trp + BisSn | 15:1 | 7.8/31.25 | 0.188^{†} | 7.8/31.25 | 0.125^{†‡} |

*62.5 μg/ml was the highest concentration tested;
**249.4 μg/ml was the highest concentration tested;
^{†}Based on the MIC/MBC of CuP being 498.8 μg/ml.
^{‡}Based on the MBC of tropolone being 125 μg/ml.

TABLE 34

| | Molecular | MIC | | MBC | |
|---|---|---|---|---|---|
| Compound | ratio | (μg/ml) | FICI | (μg/ml) | FBCI |
| Tropolone (Trp) | | 7.8 | | 62.5 | |
| Silver (I) citrate | | 2.83 | | 11.3 | |
| hydrate (AgC) | | | | | |
| Trp + AgC | 3:1 | 0.49/0.71 | 0.313 | 0.98/1.41 | 0.285 |

It can be seen that both the tropolone alone and the silver salt alone are active against C. striatum NCTC 764. Surprisingly, when the tropolone is combined with either the silver salt or the bismuth salts, the data demonstrate a synergistic (FICI & FBCI recorded at ≦0.5) antimicrobial interaction. In other words, the antibacterial activity of the tropolone appears to be enhanced by the presence of the metal salt.

### Example 15

### Activity Against C. Striatum (FIC Assays) (Hinokitiol+Copper Sulphate/Acetate)

Mixtures of hinokitiol and either copper (II) sulphate or copper (II) acetate, containing various relative proportions of the two actives, were subjected to FIC assays against C. striatum NCTC 764, as described above. The results were used to prepare FIC isobolograms. All assays were conducted in triplicate.

Representative isobolograms are shown in FIGS. 6 and 7, for copper (II) sulphate and copper (II) acetate respectively. The dashed lines indicate where overall FICIs (ie FIC_{Hinokitiol}+FIC_{copper (II) sulphate} or FIC_{Hinokitiol}+FIC_{Copper (II) acetate}) equal 1, which would indicate a purely indifferent interaction. FIGS. 6 and 7 clearly demonstrate the synergistic (FICI=0.37 & 0.3) activity of the combination of hinokitiol with either copper (II) sulphate or copper (II) acetate against C. striatum NCTC 764. This in turn indicates good anti-body odour activity for the invented combinations.

### Example 16

### Topical Anti-BO Formulations

Examples 11 to 15 show that the combination of a substituted tropone with either a copper, a bismuth or a silver salt can be active against bacteria associated with body odour. This can be of use in preparing antibacterial formulations, in particular for topical application to the skin, for either prophylactic or therapeutic use in any context where such bacteria are thought to be involved as possible sources of infection. More specifically, it can be of use in preparing formulations for use against body odour, in particular in the axillae and/or feet, again suitably for topical use.

Even in cases where a combination of an optionally substituted tropone with a copper, bismuth or silver salt has an indifferent, as opposed to synergistic, antibacterial activity compared to those of the individual compounds, this can be of considerable benefit when preparing formulations for topical use. One of the compounds may be used to replace a proportion of the other, thus lowering any side effects and/or other undesirable properties of the combination without undue loss of antibacterial activity.

A topical formulation for use in the treatment of body odour may for example be prepared by combining an optionally substituted tropone such as tropolone or hinokitiol with either copper or a copper salt such as copper sulphate or copper acetate, or with bismuth or a bismuth salt such as bismuth (III) citrate, or with silver or a silver salt such as silver (I) citrate hydrate, in a suitable fluid vehicle and optionally together with conventional additives, as described above.

The formulation may be prepared and administered using known techniques. It may for example take the form of a roll-on, spray or “stick” anti-perspirant or deodorant formulation, or of a dusting powder such as a talcum powder, or of a gel or cream or ointment. It may contain an anti-perspirant and/or deodorant agent, and/or a fragrance. It may be coated on or incorporated into a sock or shoe, or a shoe insole.

1-15. (canceled)

16. A method of treating a human or animal patient suffering from or at risk of suffering from either acne or body odour, the method involving administering to the patient a therapeutically effective amount of a formulation containing (a) tropone or a substituted tropone and (b) a metal or metal salt selected from copper, copper salts, bismuth, bismuth salts, silver, silver salts, and mixtures thereof.

17. The method of claim 16, wherein the tropone (a) is tropolone.

18. The method of claim 16, wherein the component (b) comprises a copper salt.

19. The method of claim 16, wherein the component (b) comprises a bismuth salt.

20. The method of claim 16, wherein the component (b) comprises a silver salt.

21. The method of claim 16, wherein the patient is suffering from or at risk of suffering from acne.

22. The method of claim 16, wherein the patient is suffering from or at risk of suffering from body odour.

23. The method of claim 16, wherein the formulation is administered topically.

24. A method of skin care treatment for non-therapeutic purposes, the method involving applying to the skin a formulation containing (a) tropone or a substituted tropone and (b) a metal or metal salt selected from copper, copper salts, bismuth, bismuth salts, silver, silver salts, and mixtures thereof.

25. A method for controlling the growth of a bacterium, the method comprising applying, to an area or surface which is infected or suspected to be infected or capable of becoming infected with the bacterium, a formulation containing (a) tropone or a substituted tropone and (b) a metal or metal salt selected from copper, copper salts, bismuth, bismuth salts, silver, silver salts, and mixtures thereof; and wherein the formulation is applied to a non-living area or surface.
